(19) United States
(12) Patent Application Publication
(10) Pub. No.: US 2003/0103975 A1
(43) Pub. Date: Jun. 5, 2003

(54) Modulation of angiogenesis and endothelialization

(76) Inventors: Jonathan C.R. Jones, Chicago, IL (US); Meredith Gonzales, Chicago, IL (US)

Correspondence Address: KLAUBER & JACKSON, 411 HACKENSACK AVENUE, HACKENSACK, NJ, 07601

(21) Appl. No.: 10/299,058

(22) Filed: Nov. 18, 2002

Related U.S. Application Data
(63) Continuation-in-part of application No. 09/706,235, filed on Nov. 3, 2000.
(60) Provisional application No. 60/163,199, filed on Nov. 3, 1999.

(51) Int. Cl. A61K 39/395; C12N 5/06; C07K 16/24

(52) U.S. Cl. 424/145.1; 530/388.25; 435/337

(57) ABSTRACT

A novel laminin complex is described composed of subunits of α4, β3, and γ1 laminins. Further described is a fragment of α4 laminin which binds integrin, and agents capable of modulating the binding of α4 laminin to the αvβ3 integrin receptor. Therapeutic methods are disclosed for inhibiting tumor growth by inhibiting neovascularization. Also, screening methods are disclosed for identifying agents capable of modulating angiogenesis by modulating the binding of α4 laminin to the αvβ3 integrin receptor.

### STATEMENT OF RELATED APPLICATIONS

The present application is a continuation-in-part application of U.S. Ser. No. 09/706,235 filed Nov. 3, 2000, which claims priority to U.S. S. No. 60/163,199 filed Nov. 3, 1999, the disclosures of which applications are herein specifically incorporated by reference in their entirety. Applicants claim the benefits of these application under 35 U.S.C. §§119(e) and 120.

### STATEMENT OF GOVERNMENTAL SUPPORT

The research leading to the present invention was supported, at least in part, by a grant from the National Institutes of Health, Grant Nos. GM38470, DE12328, and RO1 HL67016, and American Heart Association Grant No.0151136Z. Accordingly, the Government may have certain rights in the invention.

### FIELD OF THE INVENTION

The present invention provides agents capable of inhibiting the binding of α4 laminin to integrin, as well as a new isolated integrin complex. Further, the invention provides therapeutic methods for modulating angiogenesis, and screening methods for identifying agents capable of modulating angiogenesis.

### BACKGROUND OF THE INVENTION

Laminins, heterotrimeric molecules composed of α, β and γ subunits, are major components of basement membranes found in a variety of different tissue types. There are at least 14 laminin isoforms that regulate a variety of cellular functions including adhesion, migration, proliferation, cell survival and differentiation (see, for example, Tunggal et al. (2000) Micro. Res. Tech. 51:214-227). Although certain laminin isoforms, namely laminin 10 (α5,β1,γ1), show widespread tissue distribution, the expression of other laminin isoforms is tissue specific and tightly regulated during development. Laminins 8 (α 4, β 1, γ 1) and 9 (α 4, β 2, γ 1) are expressed by endothelial and smooth muscle cells but their functions in vivo remain unclear (Aumailley and Smyth (1998) J. Anat. 193:1-21).

Compared to α1, α2, and α 5, the α 4 subunit present in laminins 8 and 9 contains a truncated N-terminus (see, for example, Frieser et al. (1997) Eur. J. Biochem. 246:727-735). In this regard it is similar to the α 3 subunit present in laminins 5,6 and 7. However, like all other known a subunits, the α 4 laminin subunit possesses a large C-terminal G domain, consisting of 5 structurally and functionally distinct regions (G1-G5) (see, for example, Talts et al. (2000) J. Biol. Chem. 275:35192-35199). The expression of the α 4 laminin subunit is restricted to certain tissues. It is found in vascular endothelial basement membranes of brain, muscle and bone marrow, as well as the perineurium of peripheral nerves, heart, developing skeletal muscle and developing kidney (see, for example, livanainen et al. (1997) FEBS Lett. 365:183-188 and Gu et al. (1999) Blood 93:2533-2542). Indeed, the expression of α 4 laminin protein has been used as a marker of the vascularity of certain types of tumors (Niimi et al. (1997) Matrix Biol. 16:223-230; Tokida et al. (1990) J. Bio. Chem. 265:18123-18129).

### BRIEF SUMMARY OF THE INVENTION

The present invention identifies a novel adhesion structure that is involved in regulating processes that are critical for angiogenesis, including cell migration. This adhesion structure has at its core the αvβ3 integrin and the α4 laminin subunit. The present invention also provides novel methods for identifying agents/factors that modulate angiogenesis and methods of treating conditions in which angiogenesis plays a significant role.

In a first aspect, the invention provides a protein fragment of α4 laminin capable of binding the αvβ3 integrin receptor. In a more specific embodiment, the protein fragment is an amino acid sequence comprising SEQ ID NO:6, as well as homologous sequences of SEQ ID NO:6 which are capable of binding the αvβ3 integrin receptor. In another more specific embodiment, the protein fragment is an amino acid sequence having at least 75%, at least 80%, at least 85%, at least 90%, at least 95%, at least 99% homology to the amino acid sequence of SEQ ID NO:6. In a related embodiment, the invention provides an antigenic fragment of α4 laminin. In a more specific embodiment, the antigenic fragment is SEQ ID NO:6. In another embodiment, the invention provides a chimeric and/or fusion protein comprising a protein fragment of α4 laminin capable of binding the αvβ3 integrin receptor. In a more specific embodiment, the protein fragment is an amino acid sequence comprising SEQ ID NO:6, as well as homologous sequences of SEQ ID NO:6 which are capable of binding the αvβ3 integrin receptor.

In a second aspect, the invention features an agent capable of inhibiting the binding a protein fragment of α4 laminin to the αvβ3 integrin receptor. In one embodiment, the agent is an antibody raised against a protein fragment of α4 laminin capable of inhibiting the binding of α4 laminin to the αvβ3 integrin receptor. In a more specific embodiment, the antibody is raised against a protein fragment which the amino acid sequence of SEQ ID NO:6. Even more specifically, the antibody is 2A3. In another more specific embodiment, the antibody is raised against a protein fragment which is an amino acid sequence having at least 75%, at least 80%, at least 85%, at least 90%, at least 95%, at least 99% homology to the amino acid sequence of SEQ ID NO:6. In related embodiments, the agent is an antibody raised to an antigenic fragment of the amino acid sequence of SEQ ID NO:6. In a particular embodiment, the antibody is raised against a chimeric and/or fusion protein comprising that antigenic fragment. In further specific embodiments, the antibody is a monoclonal, humanized, transgenic or human antibody, or a fragment thereof. In further related embodiments, the invention provides a solid support comprising an antibody or a fragment of the antibody specific for a protein having the amino acid sequence of SEQ ID NO:6 and/or SEQ ID NO:5.

In a third aspect, the invention provides a new laminin complex, named “laminin-x”, comprising an α4 subunit, a β3 subunit, and a γ 1 subunit. In a more specific embodiment, the α4 subunit has the amino acid sequence of SEQ ID NO:2, the β3 subunit has the amino acid sequence of SEQ ID NO: 10, and the γ1 subunit has the amino acid sequence of SEQ ID NO:12.

In a fourth aspect, the present invention provides methods of inhibiting angiogenesis by administering an agent capable of inhibiting the binding of a protein fragment of α4 laminin to the αvβ3 integrin receptor. In one embodiment, the agent comprises the amino acid sequence of SEQ ID NO:6 or SEQ ID NO: 13. In further embodiments, the agent comprises an amino acid sequence having at least 75%, at least 80%, at least 85%, at least 90%, at least 95%, at least 99% homology to the amino acid sequence of SEQ ID NO:6 or SEQ ID NO: 13. In another embodiment of the method of the invention, the agent is an antibody raised against a protein fragment of α4 laminin, wherein the antibody is capable of inhibiting binding of α4 laminin or a fragment thereof to the αvβ3 integrin receptor. More specifically, the antibody is raised against a protein fragment which the amino acid sequence of SEQ ID NO:6. Even more specifically, the antibody is the “2A3” antibody described below. In another more specific embodiment, the antibody is raised against a protein fragment which is an amino acid sequence having at least 75%, at least 80%, at least 85%, at least 90%, at least 95%, at least 99% homology to the amino acid sequence of SEQ ID NO:6.

In a fifth aspect, the invention provides methods of inducing solid tumor tissue regression in an subject (e.g., a human patient), comprising administering to the subject a composition that comprises a therapeutically effective amount of an agent capable of inhibiting the binding a protein fragment of α4 laminin to the αvβ3 integrin receptor, wherein neovascularization of the solid tumor tissue is inhibited. In a specific embodiment, the method further comprises administering an anti-tumor immunotherapeutic agent and a tumor associated antigen targeting component.

In a sixth aspect, the present invention provides methods of promoting angiogenesis, comprising administering an agent capable of promoting the ability of endothelial cells to form a cell matrix junction. In one embodiment, the agent is a laminin comprising an α4 laminin subunit capable of binding integrin. In further embodiments, the laminin is laminin-8, laminin 9, and/or laminin-x. Preferably, the laminin is provided on a solid support. In one such embodiment the laminin is administered in conjunction with a graft. In a preferred embodiment of this type the laminin is administered in conjunction with a stent.

In a seventh aspect, the invention provides methods of enhancing angiogenesis in tissues containing implanted cells, comprising administering an α4 laminin subunit or a fragment thereof (e.g., either comprising or consisting essentially of the amino acid sequence of SEQ ID NO:6) that promotes the ability of endothelial cells to form a cell matrix junction.

In an eighth aspect, the invention features a screening method for identifying an agent capable of modulating the binding of α4 laminin to the αvβ3 integrin receptor, comprising determining the binding of α4 laminin to αvβ3 in the presence and absence of a test compound. A test compound which decreases α4 laminin binding to αvβ3 relative to the binding measured in the absence of the test compound is identified as an agent capable of inhibiting the binding of α4 laminin to αvβ3, and a test compound which increases the binding of α4 laminin to αvβ3 relative to the binding measured in the absence of the test compound is identified as an agent capable of increasing the binding of α4 laminin to αvβ3. Methods for determining the binding of α4 laminin to αvβ3 may be direct, e.g., measurement of adhesion of endothelial cells to α4 laminin or a fragment thereof, or indirect, e.g., determination of blood vessel development in an in vivo model. Methods for determining α4 laminin binding to αvβ3 are described in the Examples section below.

These and other aspects of the present invention will be better appreciated by reference to the following drawings and Detailed Description.

### BRIEF DESCRIPTION OF THE DRAWINGS

FIGS. 1A-I shows laminin α 4 subunit and integrin subunit localization in blood vessels. Cryostat sections of human renal carcinoma tissue were incubated with a polyclonal rabbit antiserum against the integrin β 3 (A,G) in combination with either antibody 2A3 against the α 4 laminin subunit (B) or antibody P1B5 against α3 integrin (H). In D and E, tissue was processed using antibody GoH3 against α6 integrin in combination with antibody 2A3 (E). Merged images are shown in C, F, I. Bar, 50 μm.

FIG. 2A-C. FIG. 2A shows endothelial cells adhere to varying concentrations of the α4 G domain fragment indicated. The curves are representative of three separate experiments. FIG. 2B shows cell attachment of endothelial cells to G⁹¹⁹⁻¹²⁰⁷ in the presence of control IgG or function-blocking antibodies against αvβ3 integrin (LM609), α3 integrin (P1B5), α6 integrin (GoH3), a combination of P1B5 and GoH3 or β1 integrin (6S6). LM609 was used at 25 μg/ml while all other antibodies and control IgG were used at 50 μg/ml. FIG. 2C shows endothelial cell adhesion to laminin 5 in the presence of the indicated antibodies. Values in bar graphs are expressed as mean±SD of three trials.

FIGS. 3A-D. Wells of a 96-well plate were coated with varying concentrations of G⁹¹⁹⁻¹²⁰⁷, G⁹¹⁹⁻¹⁰¹⁸ and G¹⁰¹⁶⁻¹²⁰⁷ (A) or fibronectin (B). αvβ3 integrin (5 ng/μl) was then added to, allowed to bind for 1 h at 37° C., and binding evaluated by ELISA using LM609 followed by a secondary antibody conjugated to alkaline phosphatase. FIG. 2C is a competition binding curve. Soluble αvβ3 (5 ng/μl) was added to wells coated with G⁹¹⁹⁻¹²⁰⁷ in the presence of increasing concentrations of fibronectin at 37° C. FIG. 2D shows the binding of α3β1 integrin in the presence of varying concentrations of G⁹¹⁹⁻¹²⁰⁷, G⁹¹⁹⁻¹⁰¹⁸ and G¹⁰¹⁶⁻¹²⁰⁷ Integrin binding was evaluated by ELISA using MKID2 followed by alkaline phosphatase-conjugated secondary antibody. In all studies, absorbance was measured at 405 nm. Each of the graphs is representative of at least three separate experiments.

FIG. 4A-D shows immunofluorescent microscopic photographs of HDMEC implants mixed with 0.5 ml Matrigel in the presence of 25 μg/ml of antibody 2A3 (A), 125 μg/ml of antibody 2A3 (B), 25 μg/ml of control IgG (C) or 125 μg/ml of control IgG (D). Samples were stained with anti-human type IV collagen antibody (A-D). Type IV collagen staining appears in an annular and linear organization in A-D. In E, the number of vascular structures observed in the specimens shown in A-D were quantified. Results represent mean±SD of 5 separate fields. Bar, 20 μm.

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to describe the methods and/or materials in connection with which the publications are cited.

Definitions

The terms “α4 subunit”, “α4 laminin”, and “α4 laminin subunit” are used interchangeably herein and denote a polypeptide that is a subunit of laminin-8, laminin-9, and as taught herein, laminin-x. The human α4 laminin has the amino acid sequence of SEQ ID NO:2.

As used herein, a “laminin complex” is a laminin that comprises at least three laminin subunits (e.g., α4 laminin, β3 laminin, γ1 laminin etc.). A “functional laminin complex” can bind to its receptor in cell matrix junction.

As used herein, an “α4 laminin antagonist” is a factor (e.g., a protein or fragment thereof, peptide, drug or other chemical reagent) that can hinder the ability of a laminin complex that comprises the α4 laminin subunit (e.g., laminin-8, laminin-9, and laminin-x) to bind its corresponding receptor. For example, an antibody to the α4 laminin fragment comprising the amino acid sequence of SEQ ID NO:6 is an α4 laminin antagonist. Similarly, an α4 laminin antagonist can be an α4 laminin fragment that competes with the laminin complex for its receptor.

“TrHBMEC” is a transformed human bone marrow endothelial cell and “HMVEC” is a human microvascular endothelial cell. “VMA” is vimentin-associated matrix adhesion.

As used herein a “polypeptide” is used interchangably with the term “protein” and denotes a polymer comprising two or more amino acids connected by peptide bonds. Preferably, a polypeptide is further distinguished from a “peptide” with a peptide comprising about twenty or less amino acids, and a polypeptide or protein comprising more than about twenty amino acids. A polypeptide, peptide, or fragment thereof “consisting essentially of” (or that “consists essentially of”) a specified amino acid sequence is a polypeptide, peptide, or fragment thereof that retains the general characteristics, e.g., immunogenic activity of the polypeptide, peptide, or fragment thereof having the specified amino acid sequence and is otherwise identical to that protein in amino acid sequence except it consists of plus or minus 5% or fewer, preferably plus or minus 2% or fewer, and more preferably plus or minus 1% or fewer amino acid residues. Thus, a polypeptide that consists essentially of an amino acid sequence of residues 918-1213 of SEQ ID NO:4 consists of between 281 to 311 amino acid residues, preferably 290 to 302 amino acid residues, and more preferably 293 to 299 amino acids. Preferably the additional, missing, and/or substituted amino acids are at or near the C-terminal or N-terminal portion of the protein or protein fragment. Two amino acid sequences are “substantially homologous” or “substantially similar” when greater than 25% of the amino acids are identical (preferably at least about 50%, more preferably at least about 75%, and most preferably at least about 90 or 95% identical), or greater than about 60% (preferably at least about 75%, more preferably at least about 90%, and most preferably at least about 95, 99, or 100%) are functionally identical. The sequence comparison is performed over a contiguous block of amino acid residues comprised by 4, for example. In one embodiment, selected deletions or insertions that could otherwise alter the correspondence between the two amino acid sequences are taken into account. Preferably standard computer analysis is employed for the determination that is comparable, (or identical) to that determined with an Advanced Blast search at www.ncbi.nlm.nih.gov under the default filter conditions [e.g., using the GCG (Genetics Computer Group, Program Manual for the GCG Package, Version 7, Madison, Wis.) pileup program using the default parameters].

As used herein, the terms “fusion protein”, “fusion peptide” or “fusion fragment” (when referring to a fragment of a protein) are used interchangeably and encompass “chimeric proteins and/or chimeric peptides” and fusion “intein proteins/peptides”. A fusion protein comprises at least a portion of a polypeptide (e.g., the α4 laminin) of the present invention joined via a peptide bond to at least a portion of another protein or peptide in a chimeric fusion protein. In a particular embodiment the portion of the α4 laminin is antigenic. For example, fusion proteins can comprise a marker protein or peptide, or a protein or peptide that aids in the isolation and/or purification of the α4 laminin of the present invention.

A molecule is “antigenic” when it is capable of specifically interacting with an antigen recognition molecule of the immune system, such as an immunoglobulin (antibody) or T cell antigen receptor. An antigenic polypeptide contains at least about 5, and preferably at least about 10 amino acids and more preferably about 20 amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic need not be itself immunogenic, i.e., capable of eliciting an immune response without a carrier.

General Description

The inhibition of angiogenesis can reduce the deleterious effects due to and/or contributed by the growth and/or generation of new blood vessels in diseases such as diabetic retinopathy, inflammatory diseases (either immune and non-immune inflammation), psoriasis, rheumatoid arthritis, chronic articular rheumatism, neovascular glaucoma, restenosis, capillary proliferation in atherosclerotic plaques and osteoporosis. Further, inhibition of angiogenesis can reduce the supply of blood to growing tissues, such as tumors that require neovascularization to grow beyond a few millimeters in thickness, and for the establishment of solid tumor metastases. Indeed, such disorders can also include angiofibromas, retrolental fibroplasia, hemangiomas, and Kaposi sarcoma.

U.S. Pat. No. 5,766,591 (herein specifically incorporated by reference in its entirety) has shown that the inhibition of αvβ3 effectively inhibits angiogenesis. As disclosed herein, α4 laminin has been unexpectedly found to be a ligand for αvβ3 (i.e., the corresponding receptor) thereby allowing the development of therapeutic compositions with potentially high specificity, and relatively low toxicity. Indeed, the methods of the present invention are highly selective for angiogenesis with respect to other biological processes. The specificity of the α4 laminin-αvβ3 interaction may result from unique epitope availability in newly developing blood vessels. The therapeutic methods of the instant invention allow specific targeting of newly forming blood vessels without adversely affecting mature blood vessels.

The α4 laminin subunit is a component of endothelial cell basement membranes. An antibody (2A3) against the α4 laminin G domain stains focal contact-like structures in transformed microvascular endothelial cells (TrHBMECs) and primary microvascular endothelial cells (HMVECs), provided the latter cells are activated with growth factors. The 2A3 antibody staining co-localizes with that generated by αv and β3 integrin antibodies and, consistent with this localization, TrHBMECs and HMVECs adhere to the α4 laminin subunit G domain in an αvβ3 integrin-dependent manner. The αvβ3 integrin/2A3 antibody positively-stained focal contacts are recognized by vinculin antibodies as well as by antibodies against plectin. Unusually, vimentin intermediate filaments, in addition to microfilament bundles, interact with many of the αvβ3 integrin-positive focal contacts. Since α4 laminin and αvβ3 integrin are at the core of these focal contacts, their function was investigated in cultured endothelial cells. Antibodies against these proteins inhibit branching morphogenesis of TrHBMECs and HMVECs in vitro as well as their ability to repopulate wounds in vitro. In addition, an endothelial cell matrix adhesion has been characterized which shows complex heterogeneous cytoskeleton association and whose assembly is regulated by growth factors. This structure has at its core the αvβ3 integrin and the α4 laminin subunit. Further, this adhesion structure is involved in regulating processes leading to angiogenesis, including cell migration.

Stem cells are an excellent source for cell and/or tissue replacement therapies. For example, diabetics could be treated by “replacement” pancreatic cells derived from the stem cells of the pancreas. Unfortunately, heretofore, once implanted, the replacement cells form “pseudo-organs” that die unless provided a supply of nutrients. In the normal pancreas these nutrients are provided by the extensive vascular network throughout the tissue. By coating the outer surface of the cells, cell aggregates and/or tissues to be implanted with an α4 laminin fragment of the present invention (and/or with an analog identified by the assays exemplified below), angiogenesis in the implanted cells and tissues can be enhanced and/or promoted. The coating of the cells and/or tissues to be implanted can performed by contacting the cells and/or tissues with the α4 laminin fragment prior to implantation. In one such embodiment, the cells and/or tissues are incubated (or soaked) in a solution comprising an α4 laminin fragment. Preferably, excess unbound α4 laminin fragment is removed prior to implantation by washing the cells and/or tissues with a solution that does not contain the α4 laminin fragment. In a preferred embodiment, the α4 laminin fragment has an amino acid sequence that comprises and/or consists essentially of SEQ ID NO:6.

Alternatively, the implanted cells themselves can be engineered to produce and secrete (preferably transiently) the α4 laminin or fragment thereof by molecular genetic means (as disclosed below), including through ex vivo gene therapy, or through insertion of the genetic material into a stem cell, such as an adult stem cell, an embryonic stem cell or a hematopoietic stem cell. Preferably these engineered cells also express the β laminin and the γ laminin subunits that can associate with the recombinant α4 laminin or fragment thereof so as to ensure that an ensuing laminin complex (e.g., laminin-8, laminin-9, and laminin-x) incorporates into the matrix of cells.

Laminin Proteins and Protein Fragments

Laminin-x comprises an α4 subunit, a β3 subunit, and a γ1 subunit. In the human laminin-x, the α4 subunit has the amino acid sequence of SEQ ID NO:2 and is encoded by the nucleotide sequence of SEQ ID NO: 1, the β3 subunit has the amino acid sequence of SEQ ID NO: 10 and is encoded by the nucleotide sequence of SEQ ID NO:9, and the γ1 subunit has the amino acid sequence of SEQ ID NO: 12 and is encoded by the nucleotide sequence of SEQ ID NO: 11. However, due to the degeneracy of nucleotide coding sequences, other DNA sequences that encode substantially the same amino acid sequences may be used in the practice of the present invention including those comprising conservative substitutions thereof. These include but are not limited to modified allelic genes, modified homologous genes from other species, and nucleotide sequences comprising all or portions of the laminin genes which are altered by the substitution of different codons that encode the same amino acid residue within the sequence, thus producing a silent change. Likewise, the laminin derivatives of the invention can include, but are not limited to, those containing, as a primary amino acid sequence, all or part of the amino acid sequence of a laminin including altered sequences in which functionally equivalent amino acid residues are substituted for residues within the sequence resulting in a conservative amino acid substitution. And thus, such substitutions are defined as a conservative substitution. In a particular example of this, the α4 laminin having the amino acid sequence of SEQ ID NO:4 differs from that of the naturally occurring α4 laminin having the amino acid sequence of SEQ ID NO:2 by the substitution of a single amino acid at position 918, in which a serine residue is replaced by a glycine residue.

The nucleic acids encoding laminin derivatives and analogs of the invention can be produced by various methods known in the art. The manipulations which result in their production can occur at the gene or protein level. For example, a nucleic acid encoding an α4 laminin can be produced from a native α4 laminin by any of numerous strategies known in the art (e.g., Sambrook et al. (1989)). The sequence can be cleaved at appropriate sites with restriction endonuclease(s), followed by further enzymatic modification if desired, isolated, and ligated in vitro. In the production of the gene encoding a derivative or analog of a α4 laminin, care should be taken to ensure that the modified gene remains within the same translational reading frame as the α4 laminin gene, uninterrupted by translational stop signals, in the gene region where the desired activity is encoded.

Additionally, the laminin-encoding nucleic acid sequence can be produced by in vitro or in vivo mutations, to create and/or destroy translation, initiation, and/or termination sequences, or to create variations in coding regions and/or form new restriction endonuclease sites or destroy preexisting ones, to facilitate further in vitro modification. Preferably such mutations will further enhance the specific properties of the laminin gene product. Any technique for mutagenesis known in the art can be used, including but not limited to, in vitro site-directed mutagenesis (see, for example, Hutchinson et al. (1986) Proc. Natl. Acad. Sci. USA 83:710), use of TAB® linkers (Pharmacia), etc. PCR techniques are preferred for site directed mutagenesis. A general method for site-specific incorporation of unnatural amino acids into proteins is described in Noren et al. (1989) Science 244:182-188). This method may be used to create analogs with unnatural amino acids.

Antibodies to the α4 Laminin

According to the present invention, α4 laminin as produced by a recombinant source, or through chemical synthesis, or isolated from natural sources; and derivatives or analogs thereof, including fusion proteins, may be used as an immunogen to generate antibodies that recognize α4 laminin, as exemplified below. Such antibodies include but are not limited to polyclonal, monoclonal, chimeric including humanized chimeric, single chain, Fab fragments, and a Fab expression library. The anti-α4 laminin antibodies of the invention may be cross reactive, that is, they may recognize a α4 laminin derived from a different source. Polyclonal antibodies have greater likelihood of cross reactivity. Alternatively, an antibody of the invention may be specific for a single form of a α4 laminin, and in particular a specific fragment of the α4 laminin such as the fragment exemplified below having the amino acid sequence of SEQ ID NO:6.

Thus the present invention provides compositions and uses of antibodies that are immunoreactive with α4 laminins. Such antibodies “bind specifically” to α4 laminins, meaning that they bind via antigen-binding sites of the antibody as compared to non-specific binding interactions. The terms “antibody” and “antibodies” are used herein in their broadest sense, and include, without limitation, intact monoclonal and polyclonal antibodies as well as fragments such as Fv, Fab, and F(ab′)2 fragments, single-chain antibodies such as scFv, and various chain combinations. In some embodiments, the antibodies of the present invention are humanized antibodies or human antibodies. The antibodies may be prepared using a variety of well-known methods including, without limitation, immunization of animals having native or transgenic immune repertoires, phage display, hybridoma and recombinant cell culture, and transgenic plant and animal bioreactors. Both polyclonal and monoclonal antibodies may be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Lane (eds.), Cold Spring Harbor Laboratory Press, Cold Spring Harbor, N.Y., (1988).

Various procedures known in the art may be used for the production of polyclonal antibodies to the fragment of α4 laminin comprising the amino acid sequence of SEQ If) NO:6 for example, or derivatives or analogs thereof. For the production of antibody, various host animals can be immunized by injection with the α4 laminin, fragment, or a derivative (e.g., or fusion protein) thereof, including but not limited to rabbits, mice, rats, sheep, goats, etc. In one embodiment, α4 laminin can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward α4 laminin, or analog, or derivative thereof, any technique that provides for the production of antibody molecules by continuous cell lines in culture may be used. These include but are not limited to the hybridoma technique originally developed by Kohler and Milstein (1975) Nature 256:495-497), as well as the trioma technique, the human B-cell hybridoma technique, and the EBV-hybridoma technique to produce human monoclonal antibodies. In an additional embodiment of the invention, monoclonal antibodies can be produced in germ-free animals (see, for example, PCT/US90/02545).

The monoclonal antibodies of the present invention include chimeric antibodies, e.g., “humanized” versions of antibodies originally produced in mice or other non-human species. Such humanized antibodies may be prepared by known techniques and offer the advantage of reduced immunogenicity when the antibodies are administered to humans. In fact, according to the invention, techniques developed for the production of “chimeric antibodies” (see, for example, Morrison et al. (1984) J. Bacteriol. 159:870) by splicing the genes from a mouse antibody molecule specific for the α4 laminin together with genes from a human antibody molecule of appropriate biological activity can be used; such antibodies are within the scope of this invention.

Thus, a humanized antibody is an engineered antibody that typically comprises the variable region of a non-human (e.g., murine) antibody, or at least complementarity determining regions (CDRs) thereof, and the remaining immunoglobulin portions derived from a human antibody. Procedures for the production of chimeric and further engineered monoclonal antibodies include, for example, Riechmann et al. (1988) Nature 332:323 and Winter and Harris (1993) TIBS 14:139). Such human or humanized chimeric antibodies are preferred for use in therapy of human diseases or disorders (described infra), since the human or humanized antibodies are much less likely than xenogenic antibodies to induce an immune response, in particular an allergic response, themselves.

Therefore, procedures that have been developed for generating human antibodies in non-human animals may be employed in producing antibodies of the present invention. The antibodies may be partially human or preferably completely human. For example, transgenic mice into which genetic material encoding one or more human immunoglobulin chains has been introduced may be employed. Such mice may be genetically altered in a variety of ways. The genetic manipulation may result in human immunoglobulin polypeptide chains replacing endogenous immunoglobulin chains in at least some, and preferably virtually all, antibodies produced by the animal upon immunization. Mice in which one or more endogenous immunoglobulin genes have been inactivated by various means have been prepared. Human immunoglobulin genes have been introduced into the mice to replace the inactivated mouse genes. Antibodies produced in the animals incorporate human immunoglobulin polypeptide chains encoded by the human genetic material introduced into the animal. Examples of techniques for the production and use of such transgenic animals to make antibodies (which are sometimes called “transgenic antibodies”) are described in U.S. Pat. Nos. 5,814,318, 5,569,825, and 5,545,806, which are hereby incorporated by reference in their entireties.

Hybridoma cell lines that produce monoclonal antibodies specific for the α4 laminins, or fragments thereof, of the present invention are also provided by the present invention. Such hybridomas may be produced and identified by conventional techniques. One method for producing such a hybridoma cell line comprises immunizing an animal with a polypeptide, harvesting spleen cells from the immunized animal, fusing said spleen cells to a myeloma cell line, thereby generating hybridoma cells, and identifying a hybridoma cell line that produces a monoclonal antibody that binds the polypeptide. The monoclonal antibodies produced by hybridomas may be recovered by conventional techniques.

According to the invention, techniques described for the production of single chain antibodies (U.S. Pat. Nos. 5,476,786; 5,132,405; and 4,946,778) can be adapted to produce e.g., α4 laminin-specific single chain antibodies. An additional embodiment of the invention utilizes the techniques described for the construction of Fab expression libraries (Huse et al. (1989) Science 246:1275-1281) to allow rapid and easy identification of monoclonal Fab fragments with the desired specificity for an α4 laminin fragment (e.g., the one exemplified below), or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of an α4 laminin as exemplified below, one may assay generated hybridomas for a product which binds to the α4 laminin fragment containing such an epitope and choose those which do not cross-react with α4 laminin. For selection of an antibody specific to an α4 laminin from a particular source, one can select on the basis of positive binding with 0:4 laminin expressed by or isolated from that specific source.

The foregoing antibodies can be used in methods known in the art relating to the localization and activity of the α4 laminin, e.g., for Western blotting, imaging α4 laminin in situ, measuring levels thereof in appropriate physiological samples, etc. using any of the detection techniques mentioned herein or known in the art. In a specific embodiment, antibodies that agonize or antagonize the activity of α4 laminin can be generated. Such antibodies can be tested using the assays described infra for identifying ligands.

Angiogenesis Assays

As indicated above, antibodies that bind to α4 laminin, preferably to the α4 laminin G domain, more preferably to the amino acid sequence of SEQ ID NO:6. (e.g., the 2A3 monoclonal antibody) or fragments thereof, may be used as anti-angiogenesis reagents for scientific study, in diagnostics or for treatments, such as anti-tumor treatments. As a reagent, for example, the antibody can be used as a positive control when screening for factors that block angiogenesis.

Preferred putative anti-angiogenic factors include growth factors, and proteases. Alternatively drug libraries such as those which are commercially available from most large chemical companies including Merck, GlaxoWelcome, Bristol Meyers Squib, Monsanto/Searle, Eli Lilly, and Aventis can be screened including via high throughput screening. Another source of potential drugs uses recombinant bacteriophage to produce large libraries. Using the “phage method” (see, for example, Scott and Smith (1990) Science 249:386-390), very large libraries can be constructed (10⁶-10⁸ chemical entities). A second approach uses primarily chemical methods, of which the Geysen method (1986) Molecular Immunology 23:709-715; Geysen et al. (1987) J. Immunologic Method 102:259-274) and the method of Fodor et al. (1991) Science 251:767-773) are examples. Methods of producing a mixture of peptides that can be tested as agonists or antagonists are described, for example, in U.S. Pat. Nos. 4,631,211 and 5,010,175.

In another aspect, synthetic libraries (see, for example, PCT publication WO 92/00252 and WO 9428028, each of which is incorporated herein by reference in their entireties), and the like can be used to screen for binding partners/ligands to the α4 laminin according to the present invention. Alternatively potential drugs may be synthesized de novo.

Assays such as the Matrigel assay or a cell motility assay can be employed. The matrix protein mix Matrigel (Collaborative Biomedical Products, Bedford, Mass.) may be coated as a thin gel onto the surface of the wells of a 24 well tissue culture plate (Coming, Corning N.Y.) as described below. Coated dishes can be incubated at 37° C. for 30 minutes, for example, prior to use. In a particular embodiment, approximately 6.25×10⁴ endothelial cells per cm² are plated on top of the Matrigel in each well in the presence of either the anti-α4 laminin antibody (e.g., the 2A3 described below) or a putative anti-angiogenic factor. Either primary or cell lines can be used. For example, bovine aortic endothelial cells (BAEC), calf pulmonary aortic endothelial (CPAE) cells, primary HUVEC, or HMVEC cells can be employed. Such cells are available from a number of sources including Clonetics BioWhittaker, San Diego, Calif., Cascade Biologics, Portland, Oreg., and Cell Applications, San Diego, Calif. In a preferred embodiment, neonatal dermal HMVEC cells are used. The cells can then be incubated at 37° C. for 18 hours, fixed in 2% glutaraldehyde in PBS and then photographed. Tube formation can then be scored by determining the total tube area per well. This determination can be facilitated using computer programs such as those available from Optomax Inc., Hollis, N.H., and Scanalytics, Fairfax, Va., or Metamorph (Universal Imaging Corp., Downigtown, Pa.) which provides an indication of angiogenesis (see, Cid et al. (1993) J. Clin. Invest. 91:977-985).

As noted above, motility assays also can be performed. In one such embodiment, the endothelial cells are grown to confluence in tissue culture-treated 6 well plates (Coming, Corning N.Y.) and then wounded by scraping with a pipette tip in a single stripe. The culture medium is then removed and replaced with fresh medium containing either the anti-α4 laminin antibody (e.g., the 2A3 antibody) or factor being tested. The wounded cultures are incubated at 37° C. for 18 hours, fixed in 2% glutaraldehyde in PBS and then photographed. The closure of the wound area (percent covered) can be quantified and the dimension of the wound site that remains uncovered after the defined time period gives a measure of angiogenesis inhibition. Again, the determination can be facilitated using computer programs such as those available from Scanalytics, Fairfax, Va. Motility assays may be performed using a Boyden-chamber or transwell cell migration for example.

Thus, migration toward a surface coated with a α4 laminin subunit or fragment thereof can be performed in modified Transwell chambers assay (a transwell, Haptotaxis assay, Coming Costar, Cambridge, Mass.) in the presence and/or absence of a potential modulator. The upper and lower culture compartments are separated by polycarbonate filters (8 micron pore size). The bottom surface is coated with the α4 laminin fragment. Endothelial cells (for example) are plated onto the upper surface. Cells on the filters are fixed and stained with, for example, toluidine blue at various times (e.g., 1-24 hrs after seeding). The number of cells that have migrated to the α4 laminin fragment coated surface of the filter can be determined microscopically. Alternatively the cells can be radiolabeled with ³H-thymidine. Cells that pass through the filter are harvested using trypsin/EDTA. The radioactivity is then determined using a liquid sctintillation counter. Either the cell numbers or the counts provide an indicator of angiogenesis. An increased association of the oα4 laminin or fragment thereof with the cells in the presence of the potential modulator is indicative of the potential modulator being an angiogenesis stimulator, whereas a decrease in the association can identify a potential modulator as an inhibitor.

Thus the adhesion of endothelial cells to α4 laminin fragments can be used to screen factors, e.g., potential modulators, that either inhibit or enhance angiogenesis. For example, adhesion of endothelial cells to the α4 laminin fragment can be assayed in various concentrations of the factors being screened. Preferably a known quantity of α4 laminin (or fragment thereof) would first be coated onto the well of a culture dish (e.g., a standard 96 well culture dish). A known number of endothelial cells are then plated onto the coated material in medium containing a range of concentrations of the test factor (or agent). After 1 hour the cells are washed extensively (e.g., with PBS) to remove non-adhering cells and then adherent cells can be fixed in 3.7% formaldehyde in PBS for 15 minutes at room temperature. The fixed cells are then incubated at room temperature with 0.5% crystal violet for 15 minutes and then solubilized with 1% SDS. Absorbance at 570 nm can be measured, e.g., with a Vmax plate reader (Molecular Devices, Menlo Park, Calif.), to derive a measure of the number of adherent cells. Alternatively, the determination can be performed with a digital analyzer along with an appropriate computer program to view and measure the cell area/well, by counting cells, or by DNA determinations.

Labels

The proteins and fragments thereof, including the laminins and antibodies thereto, the potential drugs identified by the present invention, as well as nucleic acids that comprise specific nucleotide sequences from a nucleic acid encoding α4 laminin can all be labeled. Suitable labels include enzymes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the receptor and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. Such labels may also be appropriate for the fragments of α4 laminin used in binding studies for example. In the instance where the label is an enzyme, detection may be accomplished by any of the presently utilized colorimetric, spectrophotometric, fluorospectrophotometric, amperometric or gasometric techniques known in the art.

Direct labels are one example of labels that can be used according to the present invention. A direct label has been defined as an entity, which in its natural state, is readily visible, either to the naked eye, or with the aid of an optical filter and/or applied stimulation, e.g. U.V. light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology (1980) 70:419-439 and in U.S. Pat. No. 4,857,453. The proteins and protein fragments of the present invention can be modified to contain a marker protein such as green fluorescent protein as described in U.S. Pat. No. 5,625,048, herein incorporated by reference in its entirety.

Suitable enzymes include, but are not limited to, alkaline phosphatase and horseradish peroxidase. Other labels for use in the invention include magnetic beads or magnetic resonance imaging labels. In one embodiment, a phosphorylation site can be created on an antibody of the invention for labeling with ³²P, e.g., as described in European Patent No. 0372707, or U.S. Pat. No. 5,459,240.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

Solid Supports

A solid phase support (or solid substrate) for use in the present invention will be inert to the reaction conditions for binding. A solid phase support for use in the present invention preferably has reactive groups in order to attach a binding partner, such as an α4 laminin or fragment thereof, or an antibody to the α4 laminin or fragment thereof, or for attaching a linker or handle which can serve as the initial binding point for any of the foregoing. As used herein, a solid phase support is not limited to a specific type of support. Rather a large number of supports are available and are known to any person with skill in the art. Solid phase supports include silica gels, resins, derivatized plastic films, glass beads, cotton, plastic beads, alumina gels, magnetic beads, membranes (including but not limited to nitrocellulose, cellulose, nylon), plastic and glass dishes or wells, etc. In a particular embodiment, the solid phase support may be a carbohydrate polymer such as SEPHAROSE, SEPHADEX, or agarose. Solid phase supports include polystyrene resin (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, California) and silica based solid phase supports such as those commercially available from Peninsula Laboratories, Inc.; and Applied Biosystems, Inc. The solid substrate can be a stent, that is preferably metallic and more preferably a Ni—Ti alloy. Stents are available from Boston Scientific Scimed, Inc., Maple Grove, Minn. 55311-1566 and Guidant Corporation, Indianapolis, Ind. 46204-5129. The solid substrate can also be a vascular graft. Such vascular grafts are available from IMPRA, Inc., Tempe, Ariz. 85281, which is a division of C.R. Bard, Murray Hill, N.J. 07974.

Methods of Treating Conditions Involving Angiogenesis

Angiogenesis plays an important role in a number of cellular processes including neovascularization of a tissue (e.g., “sprouting”), vessel enlargement and vasculogenesis. In particular, neovascularization plays a critical role in tumor growth since it is required for the transport of nutrients to the tumor. Thus, restricting neovascularization retards tumor growth, and can ultimately lead to tumor necrosis. In addition, vascularization is required for a metastatic cancer cell to exit the primary tumor and establish a secondary site. In one embodiment, the therapeutic method of the instant invention may be used in any disease or condition in which it is desirable to inhibit angiogenesis.

On the other hand, as mentioned above, stimulating angiogenesis can aid in the treatment of diseased organs to provide the nutrients to “replacement” cells for the organs that are derived from stem cells, for example. In either case, neovascularization can involve/require the α4/αvβ3 binding complex. Therefore, a second aspect of the present invention provides methods for modulating angiogenesis based on promoting or alternatively, inhibiting the formation of the α4/αvβ3 binding complex. Such methods can be used to treat and/or cure conditions that involve angiogenesis.

To implement the methods of the present invention, the therapeutic agent is preferably administered to a human, but can be administered to any animal. Thus, as can be readily appreciated by one of ordinary skill in the art, the methods and pharmaceutical compositions of the present invention are particularly suited for administration to mammals, including domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., and avian species, such as chickens, turkeys, songbirds, etc., i.e., for veterinary medical use.

One aspect of the present invention provides methods of inhibiting of angiogenesis in a tissue, (e.g., bone or muscle), an organ (e.g., liver or pancreas) and/or an organism (including humans). For example, an inflamed tissue can be treated by inhibiting neovascularization found in arthritis, chronic articular rheumatism, and psoriasis. Alternatively, a patient with diabetic retinopathy, macular degeneration or neovascular glaucoma can be treated. Moreover, conditions in which neovascularization is involved/required can also be treated by the methods disclosed herein including cancers comprising solid tumors and/or metastases, or hemangioma or angiofibroma.

Treatment of angiogenesis-related diseases, can comprise contacting a tissue in which angiogenesis is occurring, or is at risk of occurring with a composition comprising a therapeutically effective amount of an antagonist of the α4/αvβ3 complex, i.e., administering to a patient a therapeutically effective amount of a physiologically tolerable composition containing the antagonist (e.g., the therapeutic composition).

The methods of the present invention also can be employed in conjunction with other therapies such chemotherapy directed against solid tumors or to prevent/retard the establishmnent of metastases. The administration of an angiogenic inhibitor of the present invention can be performed prior to, during or after chemotherapy. Preferably, the angiogenic inhibitor of the present invention is administered together with or following a regimen of chemotherapy (i.e., at times in which the tumor tissue is being stimulated or has recently been stimulated to induce angiogenesis by the chemotherapeutic agent). Similarly, to hinder metastases, the angiogenic inhibitor of the present invention is preferably administered concurrently or shortly after the surgical removal of a solid tumor.

WO 00/47228, herein incorporated by reference in its entirety, reported that there is a synergistic effect when tumors are treated with an anti-angiogenic agent, such as those disclosed herein, in conjunction with an anti-tumor therapy (e.g., an anti-tumor antigen/cytokine fusion protein such as an antibody-Interleukin-2 (IL-2) fusion protein). Therefore, the present invention provides methods of treating tumors and metastases using therapeutic agents (including the monoclonal antibody raised against the fragment of α4 having the amino acid sequence of SEQ ID NO:6, as disclosed herein) in conjunction with immunotherapies (anti-tumor therapies).

Immunotherapeutic agents are referred to as anti-tumor antigen/cytokine fusion proteins because the agent comprises a cytokine fused with a recombinant immunoglobulin (Ig) polypeptide chain which immunoreacts with a preselected tumor-associated antigen. Anti-tumor antigen/cytokine fusion proteins have been described in U.S. Pat. No. 5,650,150 (hereby incorporated by reference in its entirety). The anti-tumor agent can be a cytokine or active fragment thereof (e.g., IL1-IL18, BDNF Accession No: 4502393, CNTF Accession No:4758020, EGF Accession No:p01133, Epo Accession No:4503589, FGF Accession No:CAB61690, F1t3L, or G-CSF Accession No:CAA27290 etc.) or a chemokine (e.g., CIO Accession No:β33861, EMF-1 Accession No:P08317 etc.) fused to an immunoglobulin that binds to a cell surface antigen. One such embodiment is a fusion protein that comprises IL-2 fused with the Ig heavy chain that immunoreacts with the tumor associated antigen GD₂. The immunotherapeutic agents can be conjugated via avidin biotin as disclosed in WO 00/47228.

Pharmaceutical Compositions

In yet another aspect of the present invention, pharmaceutical compositions comprising the therapeutic compositions of the present invention are provided. Such pharmaceutical compositions may be for administration for injection, or for oral, pulmonary, nasal or other forms of administration. In general, pharmaceutical compositions comprise effective amounts of a low molecular weight component or components, or derivative products, of the present invention together with pharmaceutically acceptable diluents, preservatives, solubilizers, emulsifiers, adjuvants and/or carriers. Such compositions include diluents of various buffer content (e.g., Tris-HCI, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. (see, e.g., Remington's Pharmaceutical Sciences (1990) 18th Ed., Mack Publishing Co., Easton, Pa. 18042: pp. 1435-1712, herein incorporated by reference in its entirety). The compositions may be prepared in liquid form, or may be in dried powder, such as lyophilized form.

Administration

According to the invention, the component or components of a therapeutic composition of the invention may be introduced parenterally, transmucosally, e.g., orally, nasally, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration. More preferably, where administration of the therapeutic composition is indicated to inhibit angiogenesis in a tumor, it may be introduced by injection into the tumor or into tissues surrounding the tumor.

The present invention also provides for conjugating targeting molecules to a therapeutic agent of the present invention. “Targeting molecule” as used herein shall mean a molecule which, when administered in vivo, localizes to desired location(s). In various embodiments, the targeting molecule can be a peptide or protein, antibody, lectin, carbohydrate, or steroid. In one embodiment, the targeting molecule is a peptide ligand of a receptor on the target cell. In a specific embodiment, the targeting molecule is an antibody. Preferably, the targeting molecule is a monoclonal antibody. In one embodiment, to facilitate crosslinking the antibody can be reduced to two heavy and light chain heterodimers, or the F(ab′)₂ fragment can be reduced, and crosslinked to the therapeutic agent via the reduced sulfhydryl. Antibodies for use as targeting molecule can be specific for cell surface antigens. The present invention further provides-for the use of other targeting molecules, such as lectins, carbohydrates, proteins and steroids.

In another embodiment, the therapeutic compound can be delivered in a vesicle, in particular a liposome. To reduce putative systemic side effects, this may be a preferred method for introducing the therapeutic composition. In yet another embodiment, the therapeutic compound can be delivered in a controlled release system. For example, a polypeptide may be administered using intravenous infusion, an implantable osmotic pump, a transdermal patch, liposomes, or other modes of administration. In one embodiment, a pump may be used. In another embodiment, polymeric materials can be used. In yet another embodiment, a controlled release system can be placed in proximity of the therapeutic target, i.e., the pancreas, thus requiring only a fraction of the systemic dose (see, e.g., Goodson (1984) in Medical Applications of Controlled Release, supra, vol. 2, pp. 115-138). Preferably, a controlled release device is introduced into a subject in proximity of the site of inappropriate immune activation or a tumor.

Other controlled release systems are discussed in the review by Langer (1990) Science 249:1527-1533). A constant supply of the therapeutic composition can be ensured by providing a therapeutically effective dose (i.e., a dose effective to induce metabolic changes in a subject) at the necessary intervals, e.g., daily, every 12 hours, etc. These parameters will depend on the severity of the disease condition being treated, other actions, such as diet modification, that are implemented, the weight, age, and sex of the subject, and other criteria, which can be readily determined according to standard good medical practice by those of skill in the art.

Similarly the dosage of the therapeutic agent will vary depending on a number of variables and can be determined by the skilled artisan, i.e., physician on a case by case basis. A therapeutically effective dosage is an amount sufficient to produce a measurable modulation of angiogenesis in the tissue being treated, i.e., an angiogenesis-inhibiting, or alternatively promoting amount. The dosage ranges for the administration depend upon the form of the therapeutic agent, and its potency (which can be determined by a variety of assays such as inhibition of angiogenesis in the CAM assay, in an in vivo rabbit eye assay, in an in vivo chimeric mouse:human assay, or by determining the effect of the agent on the binding of α4 with αvβ3, see U.S. Pat. No. 5,766,591). Modulation of angiogenesis can be measured in situ by immunohistochemistry. Appropriate dosages for comparable treatments are suggested by Brooks and Cheresh (U.S. Pat. No. 5,766,591). The dosage should be sufficiently large to achieve the desired effect but small enough to avoid any potential adverse side effects.

For example, Brooks and Cheresh (U.S. Pat. No. 5,766,591) suggest that a therapeutically effective amount of a monoclonal antibody, such as that disclosed herein, should be sufficient to achieve a plasma concentration of from about 0.01 microgram (ug) per milliliter (ml) to about 100 ug/ml, or from about 0.1 mg/kg to about 300 mg/kg, in one or more dose administrations daily, for one or several days.

Gene Therapy and Transgenic Vectors

A nucleic acid encoding α4 laminin or fragment or derivative thereof, can be introduced either in vivo, ex vivo, or in vitro in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, any tissue can be specifically targeted. Examples of particular vectors include, but are not limited to, a defective herpes virus 1 (HSV1) vector (Kaplitt et al. (1991) Molec. Cell. Neurosci. 2:320-330), an attenuated adenovirus vector, such as the vector described by Stratford-Perricaudet et al. (1992) J. Clin. Invest. 90:626-630), and a defective adeno-associated virus vector (e.g., Samulski et al. (1989) J. Virol. 63:3822-3828) including a defective adeno-associated virus vector with a tissue specific promoter (see, for example, U.S. Pat. No. 6,040,172).

In another embodiment the gene can be introduced in a retroviral vector, e.g., as described in U.S. Pat. Nos. 5,399,346; 4,650,764; 4,980,289; 5,124,263. Targeted gene delivery is described in WO 95/28494. Alternatively, the vector can be introduced in vivo by lipofection. Synthetic cationic lipids designed to limit the difficulties and dangers encountered with liposome mediated transfection can be used to prepare liposomes for in vivo transfection of a gene encoding a marker (see, for example, Mackey et al. (1988) Proc. Natl. Acad. Sci. USA 85:8027-8031).

It is also possible to introduce the vector in vivo as a naked DNA plasmid. Naked DNA vectors for gene therapy can be introduced into the desired host cells by methods known in the art, e.g., transfection, electroporation (both in vitro and in vivo), microinjection, transduction, cell fusion, DEAE dextran, calcium phosphate precipitation, use of a gene gun, or use of a DNA vector transporter (see, e.g., Wu et al. (1992) J. Biol. Chem. 267:963-967).

In a preferred embodiment of the present invention, a gene therapy vector as described above employs a transcription control sequence operably associated with the sequence for the α4 laminin or fragment thereof inserted in the vector. That is, a specific expression vector of the present invention can be used in gene therapy.

Specific Embodiments

Matrix components and integrins are believed to play an important roles in formation of blood vessels (angiogenesis), a process that occurs during normal development, during wound repair, during the female reproductive cycle and in various diseases such as cancer. These factors act by modulating endothelial cell motility as well as enabling cells to aggregate to form capillary structures. The data presented in Example 1 below indicate that vimentin-associated matrix adhesions (VMAS) may be involved in these phenomena since 2A3 antibody, raised against the α4 laminin subunit, and LM609 antibody, raised against αvβ3 integrin, both inhibit branching morphogenesis of endothelial cells and delay healing of wounded endothelial cell cultures in vitro. The idea that a matrix adhesion device which binds to the intermediate filament network of endothelial cells is involved in dynamic processes such as migration and tissue morphogenesis at first would appear to be counter-intuitive since intermediate filament binding sites at the cell surface are considered to play an essential role in stabilizing tissues. For example, although certain hemidesmosome components, such as BP230 (BPAG1) and the α6β4 integrin heterodimer, may play roles during migration leading to wound healing and metastasis, it is generally accepted that hemidesmosomes in epithelial cells are stable substrate anchor points that are present in contact-inhibited cells (Guo et al. (1995) Cell 81:233-243; Rabinovitz and Mercurio (1997) J. Cell Biol. 139:1873-1884). In contrast, the VMA is partially disassembled in contact-inhibited, presumably quiescent cells. Furthermore, whereas hemidesmosomes are disassembled when cells undergo wound healing (migrate) or are activated by growth factors, the VMA in endothelial cells is assembled under the same conditions (Goldfinger et al. (1998) supra; Jones et al. (1998) supra; Mainiero et al. (1995) EMBO 14: 4470-4481). Indeed, an array of VMAs were observed at the leading front of actively moving cells repopulating a wound site. Since these are associated with plectin and vimentin intermediate filaments, vimentin, through binding to the an matrix adhesion via plectin, could well play an active role in migration, a possibility consistent with the data of Eckes et al. (2000) J. Cell Sci. 113:2455-2462). This idea is also consistent with recent reports that both vimentin-deficient and plectin deficient cells show impaired motility (Eckes et al. (1998) J. Cell Sci. 111:1897-1907).

Example 2 describes the identification of a new laminin heterodimer secreted by endothelial cells, termed laminin-x, composed of the subunits α4, β3, and γ1.

The studies presented in Example 3 below were designed to determine integrin binding partners of the α4 laminin subunit and assess functions for the α4 laminin subunit in endothelial cells in vivo and in vitro. The results show that both the α3β1 and αvβ3 integrin can directly bind the G domain of laminin α4 subunit with high affinity. Moreover, the studies described detail complex integrin interactions with the α4 laminin and provide evidence that the α4 laminin subunit is involved in blood vessel development in an in vivo model. Specifically, the 2A3 antibody was shown to inhibit blood vessel development in vivo, while the α4 laminin subunit G⁹¹⁹⁻¹²⁰⁷ fragment (SEQ ID NO: 13) also was shown to inhibit branching morphogenesis of endothelial cells.

### EXAMPLES

The following examples are put forth so as to provide those of ordinary skill in the art with a complete disclosure and description of how to make and use the assay, screening, and therapeutic methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Structure and Function of a Vimentin-Associated Matrix Adhesion in Endothelial Cells

Cell Lines: Human microvascular endothelial cells (HMVEC) were purchased from Cascade Biologics (Portland, Oreg.) and were maintained in MED131 supplemented with microvascular growth supplement. In some experiments, HMVEC were maintained in MED131 in the absence of supplements for at least 24 hours before use. To stimulate the latter cells, basic fibroblast growth factor (bFGF, obtained from GibcoBRL Life Technologies, Gaithersburg, Md.), was added directly to the MED131 culture medium at a concentration of 5 ng/ml for at least 24 hours. Immortalized human bone marrow endothelial cells (TrHBMEC) were maintained in Dulbecco's Modified Eagle's Medium containing a final concentration of 2 mM L-glutamine, 10% fetal bovine serum and 1X RPMI vitamins (Schweitzer et al. (1997) Lab. Invest. 76:25-36). SCC12 cells were maintained in culture as described by Goldfinger et al. (1998) J. Cell Biol. 141:255-265).

Antibodies and Actin Probe: The rabbit antisera against αv integrin (AB1930), β3 integrin (AB1932) and the LM609 mouse monoclonal antibody against the αvβ3 heterodimer (MAB1976Z) were purchased from Chemicon International, Inc. (Temecula, Calif.). The monoclonal β1 blocking antibody P4C10 was obtained from Gibco BRL (Gaithersburg, Md.) (Carter et al. (1990) J. Cell Biol. 110:1387-1404). RG13 antibody against the α3 laminin subunit was described previously (Gonzales et al. (1999) Mol. Bio. Cell. 10:259-270). Mouse monoclonal antibodies specific for plectin (clone 7A8), vimentin (clone V9), and vinculin (clone hVIN-1) were purchased from Sigma Chemical Co. (St. Louis, Mo.). The α4 laminin subunit rabbit antiserum was as prepared by Miner et al. (1997) J. Cell Biol. 137:685-701) and Pierce et al. (1998) Amer. J. Respir. Cell. Mol. Biol. 19:237-244). Rhodamine-conjugated phalloidin was obtained from Molecular Probes (Eugene, Oreg.). Secondary antibodies conjugated to fluorescein, rhodamine, indodicarbocyanine (Cy5) and various sized gold particles were purchased from Jackson ImmunoResearch Labs Inc. (West Grove, Pa.).

ECMProteins and Production of Recombinant α4 Protein: Human fibronectin and mouse laminin-1 were purchased from Collaborative Research (Bedford, Mass.) and GibcoBRL (Gaithersburg, Md.) respectively. An 833 base pair CDNA fragment encoding amino acid residues 918 to 1213 of the G1/2 domains of the α4 laminin subunit was generated from TrHBMEC CDNA and sub-cloned into the pBAD TOPO TA expression vector (Invitrogen, Inc., San Diego, Calif.). This vector was then transfected into the E. coli strain LMG194 (Guzman et al. (1995) J. Bacterial. 177:4121-4130). The His tagged α4 laminin protein fragment was induced in the cells by the addition of arabinose and the fragment was purified using column chromatography (Novagen, Inc., Madison, Wis.). The purity of the recombinant protein fragment was assessed by visualizing protein samples by SDS-PAGE, and following transfer to nitrocellulose, using a His probe (Pierce, Rockford, Ill.) or a S-tag probe (Novagen, Madison, Wis.).

Primers for generating the nucleic acid encoding the 4α4-laminin antigenic fragment: (1) CCAAGCCCGT CGGATCCTGG CC (SEQ ID NO:7) and (2) CAATTT ACTCGAGCAG ACAGAAAC (SEQ ID NO:8). The nucleotides in bold were substituted to create appropriate restriction enzyme cleavage sites. GGA in SEQ ID NO:7 was substituted for AGT where indicated. The change in nucleotide sequence caused the conversion of a serine (S) to a glycine (G) as indicated below by the “G” in bold (see SEQ ID NO:6). The corresponding fragment that was not modified has the amino acid sequence of SEQ ID NO:5. TCG in SEQ ID NO:8 was substituted for TGG where indicated. This is a “silent” alteration resulting in no change in the corresponding amino acid sequence.

The α4 antigenic fragment comprised a TrxTag (100 residues) plus a His tag (6 residues) plus a Thrombin site (6 residues) plus a S tag (17 residues) plus a Enterokinase cleavage site (5 residues) plus a Polylinker (5 residues) plus the amino acid sequence (SEQ ID NO:6): GSWPAYFSIVKIERVGKHGKVFL TVPSLSSTAEEKFIKKGEFSGDDSLLDLDPEDTVFYVGGVPSNFKLPTSLNLPGFVGCLELATLNNDVI SLYNFKHIYNMDPSTSVPCARDKLAFTQSRAASYFFDGSGYAVVRDITRRGKFGQVTRFDIEVRTPA DNGLILLMVNGSMFFRLEMRNGYLHVFYDFGFSSGPVHLEDTLKKAQINDAKYHEISIIYHNDKKMI LVVDRRHVKSMDNEKMKIPFTDIYIGGAPPEILQSRALRAHLPLDINFRGCMKGFQFQKKDFNLLEQT ET (Position 1112 can also have an arginine residue (R) in place of the proline (P), as has been previously indicated in the full length sequence). The cloning vector was pET 32b from Novagen (Madison, Wis.).

Production of Monoclonal Antibody 2A3: To prepare an α4 laminin subunit antibody the recombinant α4 laminin fragment was used to immunize BALB/C mice. Spleen cells of immunized mice were fused to SP2 hybridoma cells according to standard procedures (Harlow and Lane (1988) In Antibodies: A Laboratory Manual. C. S. H. Laboratory, editor, Cold Spring Harbor, N.Y. 92-121). Populations of fused cells producing antibody against the α4 laminin fragment were identified by western blotting and then cloned three times by limiting cell dilution. One of the cloned cell lines, termed 2A3, produced an IgM class antibody. Desmos, Inc. (San Diego) prepared α 2A3 ascites fluid.

Endothelial Cell Adhesion Assays: Approximately 2×10⁵ TrHBMECs or HMVECs per cm² were plated onto uncoated or specific protein-coated wells of a 96 well plate (Sarstedt, Newton, N.C.). After 90 or 120 minutes at 37° C., the cells were washed extensively with PBS to remove non-adhering cells and then adherent cells were fixed in 3.7% formaldehyde in PBS for 15 minutes at room temperature. The fixed cells were incubated at room temperature with 0.5% crystal violet for 15 minutes and then solubilized with 1% SDS. Absorbance at 570 nm was measured with a Vmax plate reader (Molecular Devices, Menlo Park, Calif.).

Immunofluorescence: Endothelial cells were grown on glass coverslips and were fixed in 3.7% formaldehyde in PBS for 5 minutes and extracted in 0.5% Triton X-100 in PBS for 10 minutes at 4° C. to allow subsequent antibody penetration. After extensive washing in PBS, the fixed and extracted cells were incubated with primary antibodies, diluted in PBS at 37° C. in a humid chamber for at least 1 hour, washed 3 times in PBS, and then incubated with the appropriate mix of fluorochrome-conjugated secondary antibodies for an additional 1 hour at 37° C. Rhodamine-conjugated phalloidin was diluted in PBS and was incubated with the fixed and extracted cells at 37° C. for 1 hour. Stained specimens were viewed using a Zeiss LSM510 laser scanning confocal microscope (Zeiss Inc., Thoinwood, N.Y.).

Immunoelectron microscopy: Endothelial cells, maintained on glass coverslips, were fixed for 15 min in 0.5% glutaraldehyde in PBS, extracted in 0.5% Triton X-100 in PBS for 30 min at 4° C., washed in PBS and then incubated for 15 minutes in 1 μg/ml NaBH₄ in PBS. A mix of primary antibody was overlaid on the cells which were then incubated overnight at 4° C. After thorough washing, the cells on coverslips were incubated for 6 hours at room temperature in a mix of gold conjugated secondary antibodies. Following washing, the cells were prepared for electron microscopy as described by Riddelle et al. (1992) J. Cell Sci. 103:475-490). Ultrathin sections were viewed in a 100CX or 1220 JEOL electron microscope at 60 kV.

Angiogenesis Assay: Matrigel was purchased from Collaborative Biomedical Products (Bedford, Mass.) and coated as a thin gel onto the surface of the wells of a 24 well tissue culture plate (Coming, Corning N.Y.) according to the instructions of the supplier. Coated dishes were incubated at 37° C. for 30 minutes prior to use. Approximately 6.25×10⁴ cells per cm² were plated on top of the Matrigel in each well. The cells were incubated at 37° C. for 18 hours, fixed in 2% glutaraldehyde in PBS and then photographed.

In vitro Scrape Wound/Migration Assay: Endothelial cells were grown to confluence in tissue culture-treated 6 well plates (Corning, Corning N.Y.) and then wounded by scraping with a pipette tip in a single stripe. The culture medium was then removed and replaced with fresh medium. The wounded cultures were incubated at 37° C. for 18 hours, fixed in 2% glutaraldehyde in PBS and then photographed.

Protein preparations, SDS-PAGE and Western Immunoblotting: Confluent cell cultures were solubilized in sample buffer consisting of 8M urea, 1% SDS in10 mM Tris-HCL, pH 6.8, 15%—mercaptoethanol. In the case of whole cell extract preparations, DNA was sheared by sonication using a 50W Ultrasonic Processor prior to SDS-PAGE (Vibracell Sonics and Materials Inc., Danbury, Conn.). Endothelial cell matrix was prepared according to Gospodarowicz (1984) In Methods for Preparation of Media Supplements and Substrata Vol. 1, (Barnes et al., eds., Alan R. Liss, New York) with modifications detailed in Langhofer et a. (1993) J. Cell. Sci. 105: 753-764). The matrix proteins were collected from the culture dish by solubilization in the urea-SDS sample buffer. Proteins were separated by SDS-PAGE, transferred to nitrocellulose and processed for immunoblotting as described Harlow and Lane (1988) supra, pp.92-121; Kiatte et al. (1989) J. Cell Biol. 109:3377-3390; Laemmli (1970) Nature 277:680-685).

Results. Preparation of α4 laminin antibody (2A3): The localization of the α4 laminin subunit in cultured endothelial cells was first determined. To do so a monoclonal antibody probe (2A3) was prepared against a recombinant G domain fragment (amino acid residues 918 to 1213 of SEQ ID NO:4, i.e., SEQ ID NO:6) of the α4 laminin subunit. This fragment includes a portion of both the G1 and G2 subdomains of the α4 laminin. Antibody 2A3 recognizes a 250 kD protein in whole cell extracts and extracellular matrix derived from both transformed (TrHBMEC) and primary (HMVEC) endothelial cells. A similar 250 kD protein in these same preparations is recognized by a rabbit antiserum against the α4 laminin subunit (Miner et al. (1997) supra; Pierce et al. (1998) supra). In addition the molecular weight of the 2A3 reactive protein is consistent with the reported size of the α4 laminin subunit (Frieser et al. (1997) Eur. J. Biochem. 246:727-735; Gu et al. (1999) supra). A second monoclonal antibody probe (4Cl) was also prepared against the same recombinant G domain fragment, i.e., SEQ ID NO:6, and it also tested positive for the recombinant protein as well as the 250 kDa protein in the TrHBMEC extract. In marked contrast, antibodies prepared against the α3 laminin subunit show no reactivity with these protein preparations.

Immunofluorescence Analyses of Endothelial Cells: The distribution of the α4 laminin subunit in subconfluent TrHBMEC cultures was determined by confocal immunofluorescence microscopy. Antibody 2A3 stains in a focal contact-like pattern along the substratum-attached surface of the cells and co-distributes with staining generated by a polyclonal antiserum against vintegrin, a major cell surface matrix receptor expressed by endothelial cells in vitro and in vivo (Brooks et al. (1994) Science 264: 569-571; Brooks et al. (1994) Cell 79: 1157-1164; Brooks et al. (1995) J. Clin. Invest. 96:1815-1822; Varner(1997) In Regulation of Angiogenesis., Goldberg and Rosen, eds., Birhauser Verlag). In TrHBMECs, αv integrin antibody colocalizes with staining generated by probes against the αvβ3 integrin complex, the focal contact protein vinculin and plectin, a cytoskeleton cross-inking protein. Thus the vinculin-positive, plectin-positive focal contacts in TrHBMECs are enriched in αvβ3 integrin heterodimers.

If the TrHBMEC are grown to confluence, such that they become contact-inhibited, then little plectin is found along sites of cell-substrate interaction. Indeed, plectin shows primarily a filamentous staining pattern throughout the cytoplasm of the cells and does not localize extensively with αv integrin. In contrast, antibodies against αv integrin and the α4 laminin subunit as well as vinculin and the αvβ3 integrin complex (LM609) co-distribute in a focal contact-staining pattern in populations of confluent TrHBMECs.

Little, if any, basal surface staining was detected in HMVECs serum starved 24 hours prior to processing for immunocytochemistry using antibody 2A3, an antiserum against αv integrin, antibodies against the αvβ3 integrin heterodimer or antibodies against plectin. The plectin antibody shows filamentous staining in the perinuclear cytoplasm of these cells. In contrast, antibodies to vinculin generate staining of small, but clearly defined, focal contacts. However, when serum starved HMVECs were stimulated with 5 ng/ml bFGF for 24 hrs prior to processing, 2A3, αvβ3 integrin and vinculin antibodies show obvious co-localization in focal contacts that are also stained by αv antibodies. Furthermore, plectin co-distributes with αv integrin in focal contacts in the growth factor-stimulated HMVECs.

Since plectin is known to link the intermediate filament cytoskeleton to the cell surface, the organization of the vimentin cytoskeleton systems in subconfluent TrHBMECs and in HMVECs that had been stimulated with bFGF were probed. Vimentin bundles associate with a large number of αv integrin and β3 integrin antibody stained focal contact structures in both cell types. Microfilament bundles also terminate on these vimentin-associated focal contacts as shown in the triple label image where co-localized proteins appear white.

There seem to be three distinct modes of interaction of vimentin intermediate filaments with the αv integrin subunit antibody stained focal contacts. Vimentin bundles appeared to terminate at the site of focal contacts and often appeared to wrap around or loop close to the αv antibody stained focal contacts. Finally, a relatively thick vimentin bundle appeared to loop pass three different focal contacts. Vimentin bundles and filaments associate with focal contacts stained by a β3 integrin antiserum in a similar fashion. Table 1 shows quantification of the number of αv and β3 integrin antibody stained focal contacts that show vimentin association in both TrHBMECs and HMVECs as determined by double labeling studies (*Cells were co-stained with vimentin and integrin antibody probes). In both cell types over 60% of the αv and β3 integrin positive plaques show interaction with the intermediate filament cytoskeleton. The number of αv and β3 integrin containing focal contacts that associate with the vimentin cytoskeleton was quantitated in 10 cells in each line.

TABLE I
Quantification of Vimentin/Focal Contact Interactions

| CELL LINE | TrHBMECs* | HMVECs* |
|---|---|---|
| % of 3integrin positive focal contacts | 74.4% | 67.6% |
| associated with vimentin | | |
| Number of focal contacts counted in | 558 | 421 |
| 10 cells | | |
| % vintegrin positive focal contacts | 73.5% | 63.3% |
| associated with vimentin | | |
| Number of focal contacts counted in 10 cells | 245 | 308 |

To provide further evidence that the vimentin cytoskeleton associates with focal contact proteins in endothelial cells, TrHBMEC were processed for double label immunogold localization using antibody preparations against β3 integrin and vimentin. Sections were prepared both en face and perpendicular to the growth substratum. In en face sections the β3 integrin subunit occurs in clusters along the substratum attached surface of the cells as visualized with 15 nm gold particles. A filament bundle which is stained by vimentin antibodies and visualized with 5 nm gold, shows association with one of two β3 integrin aggregates in the cytoplasm. This is consistent with the fluorescence observations (Table 1). In the cross sections of the cells, 5 nm gold particles are associated with clusters of 15 nm gold particles concentrated along the substratum-attached surface of the cells.

Endothelial Cell Adhesion Assays: Since αvβ3 integrin co-localizes with α4 laminin in the endothelial cell populations endothelial cells were next assessed to determine whether they bind the α4 laminin subunit in an αvβ3 integrin-dependent manner. To do so, TrHBMECs and HMVECs were plated in uncoated wells, in wells coated with the recombinant α4 laminin G1/G2 fragment, in fibronectin-coated wells or in laminin-1 coated wells in culture plates. The proportion of attached cells was determined either after 90 minutes or 120 minutes of incubation. In the case of HMVECs, the cells were plated in the presence or absence of growth factor (bFGF at 5 ng/ml). TrHBMECs adhere much better to a surface coated with the α4 laminin fragment. Attachment of TrHBMECs to the α4 laminin subunit was significantly inhibited by the αvβ3 integrin blocking-antibody LM609 (25 μg/ml) and by antibody 2A3 (1:40 dilution). To ensure that the LM609 and 2A3 antibodies were specifically inhibiting cell adhesion to the α4 laminin fragment, their ability to perturb TrHBMEC attachment to fibronectin was determined. TrHBMEC adhesion to fibronectin is not inhibited by either LM609 (Babic et al. (1999) Mol. Cell. Biol. 19: 2958-2966) or 2A3 antibodies. [106]

HMVECs show poor adhesion to uncoated surfaces and the α4 laminin fragment-coated surfaces after being maintained in culture in the absence of growth factors for 24 hours prior to introduction into the adhesion assay. To test the impact of growth factors on attachment of HMVECs to the α4 laminin fragment, HMVECs were growth factor-stimulated for 24 hours prior to the adhesion assay. The growth factor-stimulated cells show a significant increase in their capacity to attach to the α4 laminin fragment compared with unstimulated HMVECs. Furthermore, antibodies LM609 and 2A3 inhibit the attachment of stimulated HMVECs to the α4 laminin fragment.

As a control for these studies, epithelial cell attachment to the α4 laminin fragment were assessed. SCC12 keratinocytes show no specific adherence to the α4 laminin fragment since they adhere similarly to wells coated with the α4 laminin fragment as to uncoated tissue culture plastic within 90 minutes after plating. Moreover, TrHBMECs show the same adherence to wells coated with control His-tagged recombinant proteins as they do to uncoated wells.

Angiogenesis Assays: Because αvβ3 integrin is known to play an important role in angiogenesis (Brooks et al. (1994) supra; Brooks et al. (1995) supra; Ruoslahti and Engvall (1997) J. Clin. Invest. 100:S53-S56) and because a co-localization of the α4 laminin subunit and the αv integrin in the cultured cell populations was observed, the possible function of the α4 laminin subunit in certain aspects of angiogenesis such as branching morphogenesis and cell migration of endothelial cells was analyzed (Stromblad and Cheresh (1996) Trends Cell Biol. 6:462-468). First, the Matrigel morphogenesis assay was used (Grant et al., (1989) Cell 58:933-943). TrHBMECs and HMVECs, the latter cells being stimulated with growth factors for 24 hrs, were plated onto Matrigel. After 18 hrs of incubation, even when cultured in the presence of control immunoglobulin, both cell types organize into extensive tubular arrays. However, when the endothelial cells are plated onto Matrigel in the presence of either antibody LM609 against the αvβ3 integrin complex or 2A3 antibody against the α4 laminin subunit, the formation of tubular arrays is inhibited and, in the case of cells treated with 2A3 antibody, cells appear as spheroid aggregates.

To test the role of the α4 laminin subunit in endothelial cell motility, TrHBMECs and HMVECs were first grown to confluence. After in vitro “wounding” of the cell monolayers, the cell cultures were allowed to “heal” in the presence of various antibodies or control immunoglobulins. The cells incubated in control immunoglobulin migrate to cover the wound site within 18 hrs. In contrast, in wounded cultures, treated with either antibody 2A3 or antibody LM609 against αvβ3 integrin, wound closure is incomplete after the same time interval.

These wound healing studies indicate that the matrix adhesion structure composed of a plectin/αvβ3 integrin/α4 laminin complex may be involved in migration. To assess this possibility wounded TrHBMEC cultures were prepared for double label immunofluorescence at a time when cells are actively migrating into the wound site (at about 4 hours post-wounding). The αv integrin subunit and plectin as well as α4 laminin appear to be concentrated in focal contacts at the leading edge of cells as they migrate over the wound site. In addition, vimentin intermediate filaments are associated with these focal contact-like structures.

### Example 2

### A New Laminin Heterotrimer Secreted by Endothelial Cells

Immunoprecipitation Assay: Conditioned medium from confluent 7 day cultures of endothelial cells was collected and then incubated with primary antibody overnight at 4° C. Protein G SEPHAROSE beads (GibcoBRL, Gaithersburg, Md.) or anti-mouse IgM conjugated SEPHAROSE beads (Zymed, South San Francisco, Calif.) were added to the mix and incubated for an additional hour at 4° C. The beads were collected by centrifugation and washed 5 times in TBS (10 mM Tris-HCL, pH 7.4, 145 mM NaCl) containing 1% NP40 or 0.5% Triton X-100. Proteins were eluted from the beads in SDS-PAGE sample buffer and processed for SDS-PAGE/immunoblotting as described in Example 1 above.

Results. Endothelial cells were found to secrete a novel laminin. The laminin subunits expressed by endothelial cells were analyzed by immunoblotting and immunoprecipitation. The 2A3 cα4 laminin subunit antibody primarily reacts with a protein of an approximate molecular weight of 250 kD (see Example 1) as seen by western blots of the whole cell extract and the matrix preparations of TrHBMECs and HMVECs. Polyclonal serum against the α4 laminin subunit recognizes a similar sized polypeptide. Using a panel of laminin subunit antibodies, α5, β1, β2, β3, and the γ1 laminin subunits are found in the extracts and matrix of both TrHBMECs and HMVECs, whereas the β3 laminin subunit is not. The conditioned medium of TrHBMECs was processed for immunoprecipitation (IP) using antibody probes specific for each laminin subunits. The 2A3 monoclonal antibody against the α4 subunit was used in these precipitation studies. The precipitates were subsequently prepared for western immunoblotting using the specific laminin subunit antibody probes indicated. A β3 laminin subunit antibody precipates α4 and γ1 subunits from conditioned medium of TrHBMECs. Antibody 2A3 against the α4 laminin chain precipitates the β3 and γ1 laminin but neither β1 nor β2 laminin subunits from the same conditioned medium samples. A β1 laminin subunit monoclonal antibody fails to precipitate α4 laminin. A monoclonal antibody against the α5 laminin subunit antibody precipitates both the β1 and γ1 laminin chains. A γ1 laminin subunit antibody precipitates both the β1 and β2 from endothelial cell conditioned medium. These findings indicate that the conditioned medium of TrHBMECs likely contain laminin-10 and laminin-11 composed of α5, β1, γ1, and α5, β2 and γ1 laminin subunits, respectively. The data also indicate that TrHBMECs secrete a laminin heterotrimer composed of α4, β3, and γ1 subunits. The latter complex is novel and has been named laminin-x. Interestingly, neither laminin-8 or laminin-9 (i.e., complexes of α4, β1, γ1 and α4, β2, γ1 subunits respectively) have been detected in the conditioned medium of TrHBMECs, an unexpected finding since endothelial cells have been reported previously to express both laminin-8 and laminin-9.

### Example 3

### In Vitro and In Vivo Angeiogenesis

Cell Culture. Immortalized human bone marrow endothelial cells (TrHBMEC) were maintained in DMEM containing a final concentration of 2 mM L-glutamine, 10% fetal bovine serum and 1X RPMI vitamins. (Dr. Babette Weksler, Cornell Medical School, NY and Dr. Denise Paulin, Universite Paris VII and Institute Pasteur, Paris, France) (Schweitzer et al. (1997) Lab. Invest. 76:25-36). Telomerase-immortalized human dermal microvascular endothelial cells (HDMEC) were isolated and cultured as detailed in Yang et al. (2001) Nature Biotech. 19:1-7).

Antibodies. Mouse monoclonal antibodies LM609, α3 integrin subunit (P1B5), β 1 l-integrin subunit (6S6), α3β1 integrin heterodimer (MKID2), and a β 3 integrin rabbit anti-serum (AB1932) were obtained from Chemicon International (Temecula, Calif.). The rat monoclonal α6 integrin antibody (GoH3) was purchased from Beckman Coulter (Fullerton, Calif.). 2A3, a function-blocking antibody against the G domain of laminin α4, is described above. A monoclonal antibody against human collagen type IV was obtained from Sigma Chemical Co. (St. Louis, Mo.).

Matrix proteins and Integrins. Human fibronectin and Matrigel were purchased from BD Biosciences (Bedford, Mass.) and used according to the instructions of the manufacturer. Laminin 5 was derived from conditioned medium of cultured epithelial cells (Baker et al. (1996a) Exp. Cell Res. 228:262-270). The recombinant α4 laminin, was isolated from bacterial extracts as described above and Gonzales et al. (2001) Mol. Biol. Cell. 12:85-100). The α4 laminin fragment, G⁹¹⁹⁻¹²⁰⁷ (SEQ ID. NO: 13) was generated from TrHBMEC cDNA as described in (Gonzales et al. (2001) Mol. Biol. Cell. 12:85-100) and ligated into pET32a bacterial expression vector (Novagen, Madison Wis.), in order to optimize bacterial expression for the experiments described below. The resulting α4 laminin fragment, G⁹¹⁹⁻¹²⁰⁷ (SEQ. ID. NO: 13) was recognized by the 2A3 antibody and is functionally equivalent to G⁹¹⁸⁻¹²¹³ (SEQ ID. NO: 6). In addition, an α4 laminin G1 fragment (residues 919-1018)(G⁹¹⁹⁻¹⁰¹⁸) and G2 fragment (residues 1016-1207)(G¹⁰¹⁶⁻¹²⁰⁷) were produced in bacteria as follows: cDNA generated by RT-PCR from mRNA isolated from TrHBMEC was used as template for PCR using α4 laminin subunit specific forward and reverse primers. Amplified product, digested with appropriate restriction enzymes, was ligated into the pET32a protein expression vector (Novagen, Madison Wis.) in frame with sequences encoding a 6XHis tag. Reading frame and sequence was verified by automated sequencing (Biotechnology Facility, Northwestern University). Vectors were then transfected into the E. coli strain BL21. The cells were induced to express laminin α4 fusion proteins by the addition of 1 mM isopropyl β-D-thiogalactoside (IPTG) (Fisher, N.J.) and fragments were purified using column chromatography (Novagen, Inc., Madison, Wis.). The purity of all recombinant polypeptides was assessed by visualizing protein samples by SDS-PAGE as well as by Western blotting using a His probe, following transfer of protein to nitrocellulose (Pierce, Rockford, Ill.). Soluble αvβ3 and α3β1 integrin heterodimers were purchased from Chemicon International. Their purity was routinely assessed by SDS-PAGE prior to use.

Cell Adhesion Assay. Approximately 1×10⁵ TrHBMEC were plated onto uncoated or protein-coated wells of a 96 well plate (Sarstedt, Newton, N.C.) and blocked with 1% BSA in PBS for 1 h at 37° C. After 1 h at 37° C., the cells were washed extensively with PBS to remove non-adhering cells and then adherent cells were fixed in 3.7% formaldehyde in PBS for 15 min at room temperature. The fixed cells were incubated at room temperature with crystal violet for 15 min and then solubilized with 1% SDS. Absorbance at 570 nm was measured with a Vmax plate reader (Molecular Devices, Menlo Park, Calif.). Values in the concentration-response curves were normalized to maximum cell attachment. The effective concentration (EC50) is defined as the concentration of ligand that produces half-maximal cell attachment. In certain studies integrin antibodies and control, isotype-matched immunoglobulins were added to cell suspensions for 30 mm at room temperature before the cells were plated onto substrate. In function-blocking antibody studies, values were normalized to control (100%).

ELISA Assays. Wells of 96-well non-tissue culture treated plates were coated with protein at varying concentrations for 18 h at 4° C. Each well was rinsed three times and blocked with 1% BSA in PBS for 1 h at 37° C. Soluble integrin heterodimers were diluted in binding buffer (25 mM Tris buffer, 150 nM NaCl, 1 mM MgCl2, 0.5 mM MnCl2, 0.05% BSA, pH 7.5) and added to each well for a final concentration of 5 ng/μl. After incubating for 90 min at 37° C. wells were rinsed three times in binding buffer and appropriate mouse monoclonal anti-integrin antibody was added for 1 h at 37° C. Wells were then rinsed three times in PBS and alkaline phosphatase-conjugated goat anti-mouse antibody was added to the wells for an additional 1 h at 37° C. Wells were rinsed three times in PBS and 200 μl of substrate (p-nitrophenyl phosphate (PNPP)(Sigma Chemical Co., St. Louis, Mo.), diluted in ELISA buffer to a final concentration of 1 mg/ml) was added per well. Absorbance at 405 nm was measured with a Vmax plate reader (Molecular Devices, Menlo Park, Calif.). Nonspecific binding was determined by the addition of 10 mM EDTA to binding buffer. Specific binding was obtained by subtracting nonspecific binding from total binding (total binding-nonspecific binding). In saturation binding studies, the dissociation constant (Kd) corresponds to the concentration of ligand that produces half-maximal specific binding. In competition binding studies, the inhibitory concentration (IC50) is defined as the concentration of competitor that blocks 50% of specific binding. All curves were fitted with nonlinear regression using GraphPad Prism version 3.00 (San Diego, Calif.).

Immunofluorescence Microscopy. Human renal carcinoma tissue was frozen in Tissue-Tek O.C.T. Compound (Miles, Elkhart, Ind.) and was stored at −70° C. Consecutive frozen sections of 6 μm thickness were prepared using a Tissue-Tek Cryostat at −20° C. and placed on slides. Sections were extracted in acetone at −20° C. for 5 min and then air-dried. Matrigel implants were fixed in 10% buffered formalin, embedded in paraffin and sectioned. Sections were deparaffinized and antigens retrieved in 10 mM citric acid (pH 6.0) by microwaving twice for 7 mm. Tissue sections were incubated with primary antibodies, diluted in PBS, at 37° C. in a humid chamber for at least 1 h, washed 3 times in PBS, and then incubated with the appropriate mix of fluorochrome-conjugated secondary antibodies for an additional 1 h at 37° C. Stained specimens were viewed using a Zeiss 9 LSM5 10 laser scanning confocal microscope or Zeiss Axioskop microscope as indicated (Zeiss Inc., Thomwood, N.Y.).

SDS-PAGE and Western Blotting. Matrix proteins and integrins were separated on 7.5-12% SDS-polyacrylamide gels following standard procedures (Laemmli (1970) supra). Gels were either stained or separated proteins were transferred to nitrocellulose which was subsequently processed for Western blotting as previously described (Harlow and Lane (1988) supra; Klatte et al. (1989) supra).

In Vivo Angiogenesis Assay. Approximately 1×10⁶ immortalized HDMEC-GT cells were mixed with 0.5 ml of Matrigel on ice in the presence of either antibody 2A3 or control ascites immunoglobulin and the mixture was implanted into the ventral midline thoracic tissue of 4-6 week old male SCID mice following procedures outlined in Yang et al. (2001) supra. The implants were surgically removed after 1 week, sectioned, and assayed for tubule formation with anti-human type IV Collagen IgG. Immunoreactive signals for type IV collagen were seen as annular and linear structures. Two separate 10X random fields per tissue section were taken and the number of annular structures were counted and averaged.

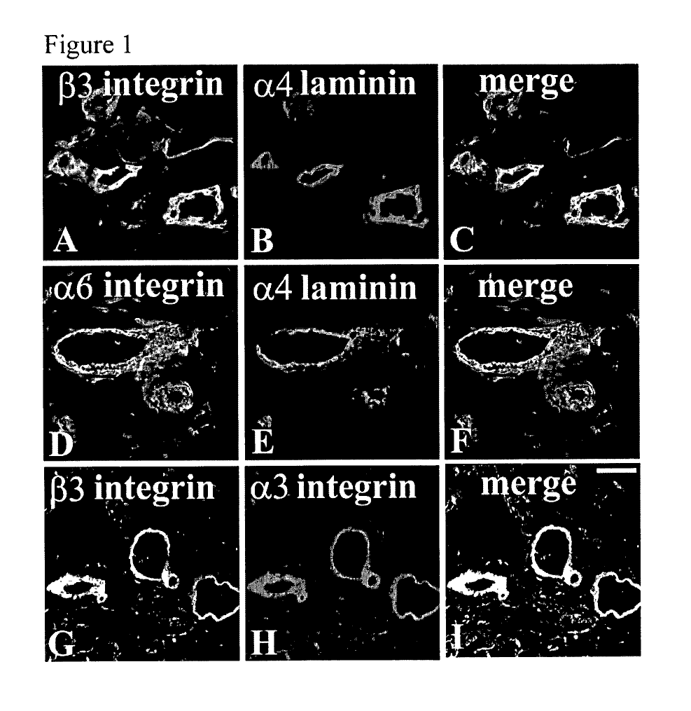

Results. In cryosections of renal carcinoma tissue that possess an extensive vasculature, 2A3 antibodies generated an intense stain along the basement membrane zone of blood vessels, a site rich in α3,α6 and β 3 subunit-containing integrins (FIG. 1). The co-distribution of the α4 laminin subunit with α3 and α6 integrin along the site of endothelial cell-basement membrane zone interaction is consistent with data indicating that cells interact with laminins 8 and 9 via α3β1 and α6β1 integrin (Fujiwara et al. (2001) J. Biol. Chem. 276:17550-17558).

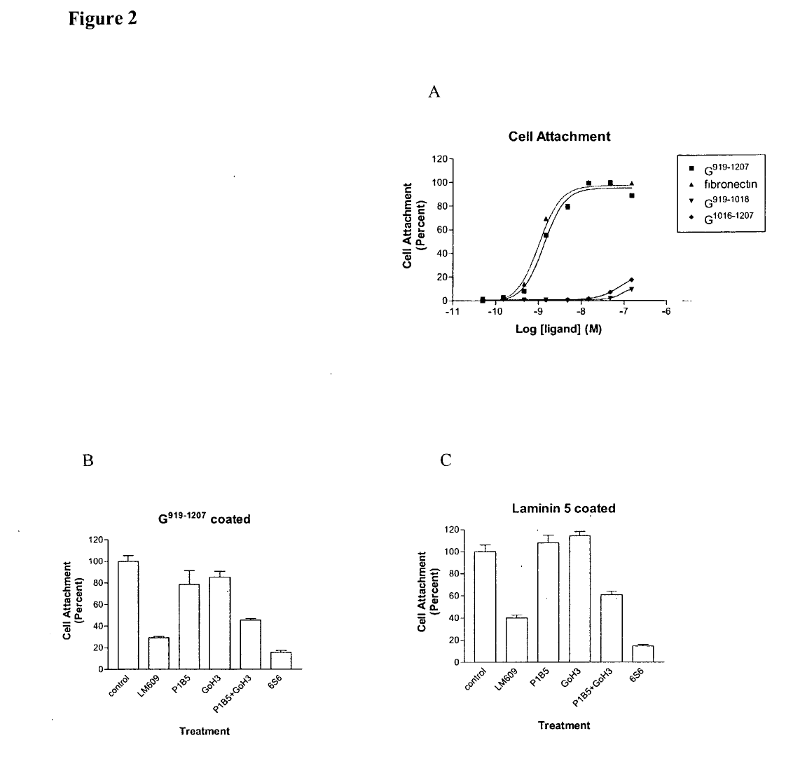

Endothelial cell adhesion to a number of α⁴ G domain fragments was evaluated. Cells attached to residues G⁹¹⁹⁻¹²⁰⁷ in a concentration-dependent manner with cell binding being half maximal (EC50) at 1.4 nM (FIG. 2A). Cell attachment to fibronectin produced a similar concentration-response curve with an EC50 of 1.0 nM. In contrast, both G⁹¹⁹⁻¹⁰¹⁸ and G¹⁰¹⁶⁻¹²⁰⁷ fragments were poor ligands for cell attachment and cell attachment failed to reach half maximal even at 100 nM ligand concentration.

The involvement of integrin in endothelial cell adhesion to G⁹¹⁹⁻¹²⁰⁷ was investigated. Endothelial cells showed maximal binding to wells coated with 100 nM concentration of G⁹¹⁹⁻¹²⁰⁷ (FIG. 2A). Antibody LM609, which perturbs the function of the α v β3 integrin, inhibited endothelial cell adhesion to G⁹¹⁹⁻¹²⁰⁷ by about 70% while 6S6, an integrin β1 function-blocking antibody, inhibited cell adhesion by approximately 84% compared to control IgG treated cells (FIG. 2B). The latter result is contrary to our previous report where we showed that antibody β4C10 against the β1 integrin failed to inhibit endothelial cell adhesion to the α4 laminin G domain (Gonzales et al. (2001) supra). To resolve the potential role of β 1 containing integrins in endothelial cell attachment to the α4 laminin subunit, we examined the effects of antibodies that functionally inhibited α6(β 1) and α 3(β 1) integrin, namely GoH3 and P1B5 on adhesion of endothelial cells to G⁹¹⁹⁻¹²⁰⁷ The α3 integrin antibody, P1B5, when used in combination with GoH3, the α 6 integrin antibody, inhibited cell adhesion by more than 54% (FIG. 2B). It should be noted that these same antibodies when used individually have minimal impact on endothelial cell adhesion to G⁹¹⁹⁻¹²⁰⁷, suggesting that the function of α3 and α6 subunit-containing integrins is in some way coupled in endothelial cells. Together these data indicate an involvement of both α6β1 and α3β1 in adhesion to the G domain of the α4 laminin subunit, a finding consistent with a previous report (Fujiwara et al. (2001) supra). However, these data also indicate that the αvβ3 integrin subunit plays a role in endothelial cell-α4 laminin subunit interaction. One intriguing aspect of the above results is that α v β3 integrin fails to “compensate” in mediating binding to G⁹¹⁹⁻¹²⁰⁷ when the α3β1 and α6β1 integrin heterodimers are functionally inhibited and vice versa. One possible explanation for our results is that in endothelial cells the activity of one integrin may modulate ligand binding of another via a process that is termed transmodulation (Hodivala-Dilke et al. (1998) J. Cell Biol. 142: 1357-1369). To test this possibility we assayed endothelial cell adhesion to laminin 5, a ligand for α3β1 but not for αvβ3, in the presence of antibodies that functionally perturb either the αvβ3 or α3β1 integrin heterodimer (FIG. 2C). As would be expected, endothelial cell adhesion to laminin 5 was inhibited by 40% when treated with a combination of antibodies against the α3 integrin subunit and α6 integrin subunits and by more than 80% by antibody 6S6, a function-blocking antibody against β1 integrin. However, in addition, it was also inhibited by 60% when endothelial cells were treated with antibody LM609 against the αvβ3 integrin. This indicates that functional perturbation of the αvβ3 integrin has a transmodulating, in this case inhibitory, impact on α3β1 integrin/laminin 5 interaction.

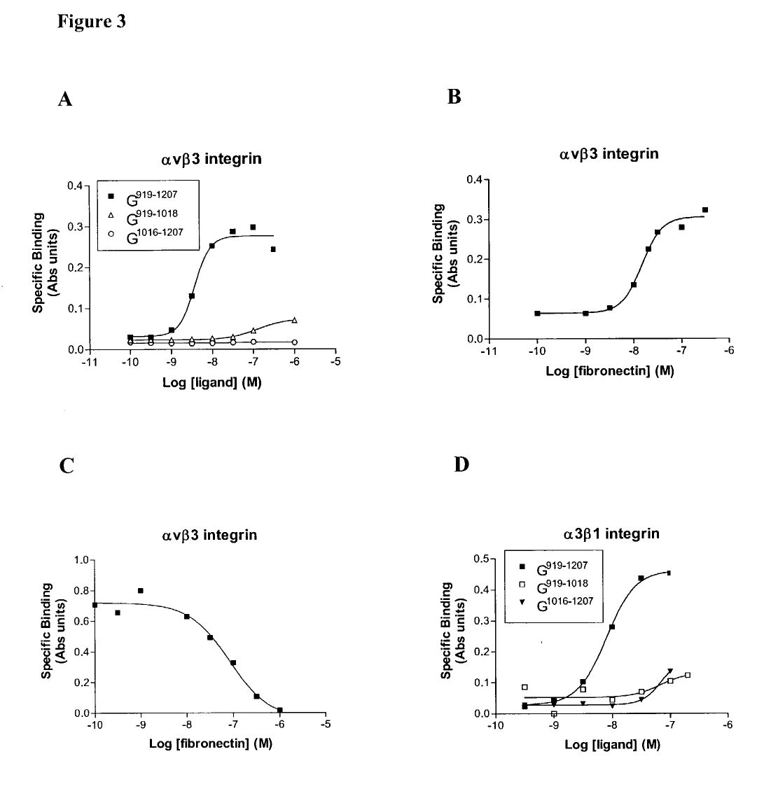

The direct binding of integrins to α4 laminin was studied with solid-phase saturation binding experiments using purified integrin and laminin proteins. To date, we have restricted our analyses to αvβ3 and α3β1 integrin binding to ligand since we have been unable to obtain appropriately pure α6β1 integrin to use in our assays. Nevertheless, soluble αvβ3 bound G⁹¹⁹⁻¹²⁰⁷ in a concentration-dependent fashion and binding is saturable (FIG. 3A). The observed dissociation constant (Kd) was 4.0 nM. This is comparable to the dissociation constant when αv β3 integrin bound fibronectin, a known ligand for this integrin heterodimer (Kd, 15 nM) (FIG. 3B) (Eliceiri and Cheresh (1999) J. Clin. Invest. 103:1227-1230). Moreover, fibronectin was able to compete with G⁹¹⁹⁻¹²¹⁷ for binding to αvβ3 in a concentration-dependent fashion with a measured IC50 of 84 nM, suggesting that fibronectin and G⁹¹⁹⁻¹²⁰⁷ bound to a similar or nearby site on the αvβ3 integrin molecule (FIG. 3C). α3β1 integrin also bound to G⁹¹⁹⁻¹²⁰⁷ with a Kd of 7.3 nM (FIG. 3D). No significant binding of α3β1 or αvβ3 to G⁹¹⁹⁻¹²⁰⁷ was detected in the presence of 10 mM EDTA (data not shown). Furthermore, α v β 3 and α3β1 integrin bound poorly to both G⁹¹⁹⁻¹⁰¹⁸ and G¹⁰¹⁶⁻¹²⁰⁷ (FIGS. 3A,D).

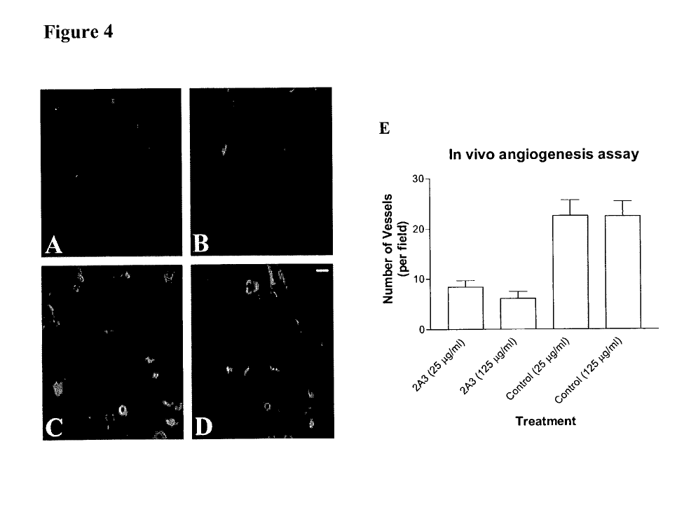

The involvement of the α4 laminin subunit in blood vessel formation assembly was investigated in vivo using a mouse-human chimeric model in which human endothelial cells are injected into SCID mice in Matrigel. After about 7 days, the human cells assembled into blood vessels that can be identified and quantified using a marker of basement membrane assembly, namely an antibody probe specific for human collagen type IV (FIG. 4A-D). Blood vessel development was evaluated by collagen IV staining under conditions where antibody 2A3 against the α4 G domain or isotype matched control immunoglobulin were added to the cell-Matrigel mix prior to injection into the SCID mice. As can be seen in FIG. 4A and B, there was a significant decrease in collagen IV antibody staining in the 2A3 antibody treated cells compared with endothelial cells treated with control immunoglobulin (FIGS. 4C,D). These results were quantified in FIG. 4E.

1. An antigenic fragment of α4 laminin comprising the amino acid sequence of SEQ ED NO:6.

2. A chimeric and/or fusion protein comprising the antigenic fragment of claim 1.

3. An antibody to the antigenic fragment of claim 1.

4. The antibody of claim 3, selected from the group consisting of a monoclonal antibody, a humanized antibody, a transgenic antibody, and a human antibody.

5. The antibody of claim 4, which is 2A3.

6. A α4 laminin function-blocking antibody or a fragment thereof.

7. The antibody of claim 6, selected from the group consisting of a monoclonal antibody, a humanized antibody, a transgenic antibody, and a human antibody.

8. The antibody of claim 7, which is 2A3.

9. A cell line that produces the antibody of claim 6.

10. An isolated laminin complex, laminin-x, comprising an α4 subunit, a β3 subunit, and a γ1 subunit.

11. A method of modulating angiogenesis, comprising administering an agent capable of modulating binding of α4 laminin to integrin.

12. The method of claim 11, wherein the modulation is an inhibition of angiogenesis.

13. The method of claim 12, wherein the agent is an antibody raised against an antigenic fragment of α4 laminin.

14. The method of claim 13, wherein the antigenic fragment of α4 laminin comprises the amino acid sequence of SEQ ID NO:6.

15. The method of claim 13, wherein the antibody is 2A3.

16. The method of claim 13, wherein angiogenesis is inhibited in newly developing blood vessels.

17. The method of claim 16, wherein the blood vessels are developing in a tumor.

18. The method of claim 12, wherein the agent is a fragment of α4 laminin.

19. The method of claim 18, wherein the fragment is a sequence having at least 75% homology to SEQ ID NO: 6 or SEQ ID NO: 13.

20. The method of claim 19, wherein the fragment is SEQ ID NO: 13.

21. A method of inducing solid tumor tissue regression in a subject, comprising administering to the subject a composition comprising a therapeutically effective amount of the antibody of claim 6, wherein neovascularization of the solid tumor tissue is inhibited.

22. The method of claim 15, further comprising administering an anti-tumor immunotherapeutic agent and a tumor associated antigen targeting component.

23. The method of claim 11, wherein the modulation is an enhancement of angiogenesis.


SEQUENCE LISTING (13 sequences)

SEQ ID NO: 1 - DNA, 5781 bp, Homo sapien
  N in position 3335 can either be a G/C.
atggctttgagctcagcctggcgctcggttctgcctctgtggctcctctggagcgctgcctgctcccgcgccgcgtccggggacgacaacgcttttccttttgacattgaagggagctcagcggttggcaggcaagacccgcctgagacgagcgaaccccgcgtggctctgggacgcctgccgcctgcggccgagaaatgcaatgctggattctttcacaccctgtcgggagaatgtgtgccctgcgactgtaatggcaattccaacgagtgtttggacggctcaggatactgtgtgcactgccagcggaacacaacaggagagcactgtgaaaagtgtctggatggttatatcggagattccatcaggggagcaccccaattctgccagccgtgcccctgtcccctgccccacttggccaattttgcagaatcctgctataggaaaaatggagctgttcggtgcatttgtaacgaaaattatgctggacctaactgtgaaagatgtgctcccggttactatggaaaccccttactcattggaagcacctgtaagaaatgtgactgcagtggaaattcagatcccaacctgatctttgaagattgtgatgaagtcactggccagtgtaggaattgcttacgcaacaccaccggattcaagtgtgaacgttgcgctcctggctactatggggacgccaggatagccaagaactgtgcagtgtgcaactgcgggggaggcccatgtgacagtgtaaccggagaatgcttggaagaaggttttgaaccccctacaggctgtgataagtgcgtctgggacctgactgatgacctgcggttagcagcgctctccatcgaggaaggcaaatccggggtgctgagcgtatcctctggggccgccgctcataggcacgtgaatgaaatcaacgccaccatctacctcctcaaaacaaaattgtcagaaagagaaaaccaatacgccctaagaaagatacaaatcaacaatgctgagaacacgatgaaaagccttctgtctgacgtagaggaattagttgaaaaggaaaatcaagcctccagaaaaggacaacttgttcagaaggaaagcatggacaccattaaccacgcaagtcagctggtagagcaagcccatgatatgagggataaaatccaagagatcaacaacaagatgctctattatggggaagagcatgaacttagccccaaggaaatctctgagaagctggtgttggcccagaagatgcttgaagagattagaagccgtcaaccatttttcacccaacgggagctcgtggatgaggaggcagatgaggcttacgaactactgagccaggctgagagctggcagcggctgcacaatgagacccgcactctgtttcctgtcgtcctggagcagctggatgactacaatgctaagttgtcagatctccaggaagcacttgaccaggcccttaaccatgtcagggatgccgaagacatgaacagggccacagcagccaggcagcgggaccatgagaaacaacaggaaagagtgagggaacaaatggaagtggtgaacatgtctctgagcacatctgcggactctctgacaacacctcgtctaactctttcagaacttgatgatataataaagaatgcgtcagggatttatgcagaaatagatggagccaaaagtgaactacaagtaaaactatctaacctaagtaacctcagccatgatttagtccaagaagctattgaccatgcacaggaccttcaacaagaagctaatgaattgagcaggaagttgcacagttcagatatgaacgggctggtacagaaggctttggatgcatcaaatgtctatgaaaatattgttaattatgttagtgaagccaatgaaacagcagaatttgctttgaacaccactgaccgaatttatgatgcggtgagtgggattgatactcaaatcatttaccataaagatgaaagtgagaacctcctcaatcaagccagagaactgcaagcaaaggcagagtctagcagtgatgaagcagtggctgacactagcaggcgtgtgggtggagccctagcaaggaaaagtgcccttaaaaccagactcagtgatgccgttaagcaactacaagcagcagagagaggggatgcccagcagcgcctggggcagtctagactgatcaccgaggaagccaacaggacgacgatggaggtgcagcaggccactgcccccatggccaacaatctaaccaactggtcacagaatcttcaacattttgactcttctgcttacaacactgcagtgaactctgctagggatgcagtaagaaatctgaccgaggttgtccctcagctcctggatcagcttcgtacggttgagcagaagcgacctgcaagcaacgtttctgccagcatccagaggatccgagagctcattgctcagaccagaagtgttgccagcaagatccaagtctccatgatgtttgatggccagtcagctgtggaagtgcactcgagaaccagtatggatgacttaaaggccttcacgtctctgagcctgtacatgaaaccccctgtgaagcggccggaactgaccgagactgcagatcagtttatcctgtacctcggaagcaaaaacgccaaaaaagagtatatgggtcttgcaatcaaaaatgataatctggtatacgtctataatttgggaactaaagatgtggagattcccctggactccaagcccgtcagttcctggcctgcttacttcagcattgtcaagattgaaagggtgggaaaacatggaaaggtgtttttaacagtcccgagtctaagtagcacagcagaggaaaagttcattaaaaagggggaattttcgggagatgactctctgctggacctggaccctgaggacacagtgttttatgttggtggagtgccttccaacttcaagctccctaccagcttaaacctgcctggctttgttggctgcctggaactggccactttgaataatgatgtgatcagcttgtacaactttaagcacatctataatatggacccctccacatcagtgccatgtgcccgagataagctggccttcactcagagtcgggctgccagttacttcttcgatggctccggttatgccgtggtgagagacatcacaaggagagggaaatttggtcaggtgactcgctttgacatagaagttcgaacaccagctgacaacggccttattctcctgatggtcaatggaagtatgtttttcagactggaaatgcgcaatggttacctacatgtgttctatgattttggattcagcagtggccntgtgcatcttgaagatacgttaaagaaagctcaaattaatgatgcaaaataccatgagatctcaatcatttaccacaatgataagaaaatgatcttggtagttgacagaaggcatgtcaagagcatggataatgaaaagatgaaaataccttttacagatatatacattggaggagctcctccagaaatcttacaatccagggccctcagagcacaccttcccctagatatcaacttcagaggatgcatgaagggcttccagttccaaaagaaggacttcaatttactcgagcagacagaaaccctgggagttggttatggatgcccagaagactcacttatatctcgcagagcatatttcaatggacagagcttcattgcttcaattcagaaaatatctttctttgatggctttgaaggaggttttaatttccgaacattacaaccaaatgggttactattctattatgcttcagggtcagacgtgttctccatctcactggataatggtactgtcatcatggatgtaaagggaatcaaagttcagtcagtagataagcagtacaatgatgggctgtcccacttcgtcattagctctgtctcacccacaagatatgaactgatagtagataaaagcagagttgggagtaagaatcctaccaaagggaaaatagaacagacacaagcaagtgaaaagaagttttacttcggtggctcaccaatcagtgctcagtatgctaatttcactggctgcataagtaatgcctactttaccagggtggatagagatgtggaggttgaagatttccaacggtatactgaaaaggtccacacttctctttatgagtgtcccattgagtcttcaccattgtttctcctccataaaaaaggaaaaaatttatccaagcctaaagcaagtcagaataaaaagggagggaaaagtaaagatgcaccttcatgggatcctgttgctctgaaactcccagagcggaatactccaagaaactctcattgccacctttccaacagccctagagcaatagagcacgcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgctttgaaggccccatggaaacaggaacttacttttcaacagaaggaggatacgtggttctagatgaatctttcaatattggattgaagtttgaaattgcatttgaagtccgtcccagaagcagttccggaaccctggtccacggccacagtgtcaatggggagtacctaaatgttcacatgaaaaatggacaggtcatagtgaaagtcaataatggcatcagagatttttccacctcagtaacacccaagcagagtctctgtgatggcagatggcacagaattacagttattagagattctaatgtggttcagttggatgtggactctgaagtgaaccatgtggttggacccctgaatccaaaaccaattgatcacagggagcctgtgtttgttggaggtgttccagaatctctactgacaccacgcttggcccccagcaaacccttcacaggctgcatacgccactttgtgattgatggacacccagtgagcttcagtaaagcagccctggtcagcggcgccgtaagcatcaactcctgtccagcagcctgacatgacagagcacagctgcccaaatacaaagttctttagagcactgaaagaaacacaaagccagccaggaggaacagtaactcttccttcgggtggaagctttcatcgagttgaacaggacttaaacgaatcatcagggaccggatatttcttatttctcatttggattcttaaccttgaatccaaagtgtctgcaatggacaacaattgaaggagaggcaaacttacttgtattgagagcacacgcaattcctactggtgaaattactgtttctgtttctaataaaatagaagggattccaaataaaaaaaaaaaaaaaaaaaaaaaaa

SEQ ID NO: 2 - protein, 1816 aa, Homo sapien
  X in position 1112 = Arg/Pro
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNHVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSSGXVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 3 - DNA, 5781 bp, Homo sapien
  The N in position 3335 can either be a G/C.
atggctttgagctcagcctggcgctcggttctgcctctgtggctcctctggagcgctgcctgctcccgcgccgcgtccggggacgacaacgcttttccttttgacattgaagggagctcagcggttggcaggcaagacccgcctgagacgagcgaaccccgcgtggctctgggacgcctgccgcctgcggccgagaaatgcaatgctggattctttcacaccctgtcgggagaatgtgtgccctgcgactgtaatggcaattccaacgagtgtttggacggctcaggatactgtgtgcactgccagcggaacacaacaggagagcactgtgaaaagtgtctggatggttatatcggagattccatcaggggagcaccccaattctgccagccgtgcccctgtcccctgccccacttggccaattttgcagaatcctgctataggaaaaatggagctgttcggtgcatttgtaacgaaaattatgctggacctaactgtgaaagatgtgctcccggttactatggaaaccccttactcattggaagcacctgtaagaaatgtgactgcagtggaaattcagatcccaacctgatctttgaagattgtgatgaagtcactggccagtgtaggaattgcttacgcaacaccaccggattcaagtgtgaacgttgcgctcctggctactatggggacgccaggatagccaagaactgtgcagtgtgcaactgcgggggaggcccatgtgacagtgtaaccggagaatgcttggaagaaggttttgaaccccctacaggctgtgataagtgcgtctgggacctgactgatgacctgcggttagcagcgctctccatcgaggaaggcaaatccggggtgctgagcgtatcctctggggccgccgctcataggcacgtgaatgaaatcaacgccaccatctacctcctcaaaacaaaattgtcagaaagagaaaaccaatacgccctaagaaagatacaaatcaacaatgctgagaacacgatgaaaagccttctgtctgacgtagaggaattagttgaaaaggaaaatcaagcctccagaaaaggacaacttgttcagaaggaaagcatggacaccattaaccacgcaagtcagctggtagagcaagcccatgatatgagggataaaatccaagagatcaacaacaagatgctctattatggggaagagcatgaacttagccccaaggaaatctctgagaagctggtgttggcccagaagatgcttgaagagattagaagccgtcaaccatttttcacccaacgggagctcgtggatgaggaggcagatgaggcttacgaactactgagccaggctgagagctggcagcggctgcacaatgagacccgcactctgtttcctgtcgtcctggagcagctggatgactacaatgctaagttgtcagatctccaggaagcacttgaccaggcccttaaccatgtcagggatgccgaagacatgaacagggccacagcagccaggcagcgggaccatgagaaacaacaggaaagagtgagggaacaaatggaagtggtgaacatgtctctgagcacatctgcggactctctgacaacacctcgtctaactctttcagaacttgatgatataataaagaatgcgtcagggatttatgcagaaatagatggagccaaaagtgaactacaagtaaaactatctaacctaagtaacctcagccatgatttagtccaagaagctattgaccatgcacaggaccttcaacaagaagctaatgaattgagcaggaagttgcacagttcagatatgaacgggctggtacagaaggctttggatgcatcaaatgtctatgaaaatattgttaattatgttagtgaagccaatgaaacagcagaatttgctttgaacaccactgaccgaatttatgatgcggtgagtgggattgatactcaaatcatttaccataaagatgaaagtgagaacctcctcaatcaagccagagaactgcaagcaaaggcagagtctagcagtgatgaagcagtggctgacactagcaggcgtgtgggtggagccctagcaaggaaaagtgcccttaaaaccagactcagtgatgccgttaagcaactacaagcagcagagagaggggatgcccagcagcgcctggggcagtctagactgatcaccgaggaagccaacaggacgacgatggaggtgcagcaggccactgcccccatggccaacaatctaaccaactggtcacagaatcttcaacattttgactcttctgcttacaacactgcagtgaactctgctagggatgcagtaagaaatctgaccgaggttgtccctcagctcctggatcagcttcgtacggttgagcagaagcgacctgcaagcaacgtttctgccagcatccagaggatccgagagctcattgctcagaccagaagtgttgccagcaagatccaagtctccatgatgtttgatggccagtcagctgtggaagtgcactcgagaaccagtatggatgacttaaaggccttcacgtctctgagcctgtacatgaaaccccctgtgaagcggccggaactgaccgagactgcagatcagtttatcctgtacctcggaagcaaaaacgccaaaaaagagtatatgggtcttgcaatcaaaaatgataatctggtatacgtctataatttgggaactaaagatgtggagattcccctggactccaagcccgtcggatcctggcctgcttacttcagcattgtcaagattgaaagggtgggaaaacatggaaaggtgtttttaacagtcccgagtctaagtagcacagcagaggaaaagttcattaaaaagggggaattttcgggagatgactctctgctggacctggaccctgaggacacagtgttttatgttggtggagtgccttccaacttcaagctccctaccagcttaaacctgcctggctttgttggctgcctggaactggccactttgaataatgatgtgatcagcttgtacaactttaagcacatctataatatggacccctccacatcagtgccatgtgcccgagataagctggccttcactcagagtcgggctgccagttacttcttcgatggctccggttatgccgtggtgagagacatcacaaggagagggaaatttggtcaggtgactcgctttgacatagaagttcgaacaccagctgacaacggccttattctcctgatggtcaatggaagtatgtttttcagactggaaatgcgcaatggttacctacatgtgttctatgattttggattcagcagtggccntgtgcatcttgaagatacgttaaagaaagctcaaattaatgatgcaaaataccatgagatctcaatcatttaccacaatgataagaaaatgatcttggtagttgacagaaggcatgtcaagagcatggataatgaaaagatgaaaataccttttacagatatatacattggaggagctcctccagaaatcttacaatccagggccctcagagcacaccttcccctagatatcaacttcagaggatgcatgaagggcttccagttccaaaagaaggacttcaatttactcgagcagacagaaaccctgggagttggttatggatgcccagaagactcacttatatctcgcagagcatatttcaatggacagagcttcattgcttcaattcagaaaatatctttctttgatggctttgaaggaggttttaatttccgaacattacaaccaaatgggttactattctattatgcttcagggtcagacgtgttctccatctcactggataatggtactgtcatcatggatgtaaagggaatcaaagttcagtcagtagataagcagtacaatgatgggctgtcccacttcgtcattagctctgtctcacccacaagatatgaactgatagtagataaaagcagagttgggagtaagaatcctaccaaagggaaaatagaacagacacaagcaagtgaaaagaagttttacttcggtggctcaccaatcagtgctcagtatgctaatttcactggctgcataagtaatgcctactttaccagggtggatagagatgtggaggttgaagatttccaacggtatactgaaaaggtccacacttctctttatgagtgtcccattgagtcttcaccattgtttctcctccataaaaaaggaaaaaatttatccaagcctaaagcaagtcagaataaaaagggagggaaaagtaaagatgcaccttcatgggatcctgttgctctgaaactcccagagcggaatactccaagaaactctcattgccacctttccaacagccctagagcaatagagcacgcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgctttgaaggccccatggaaacaggaacttacttttcaacagaaggaggatacgtggttctagatgaatctttcaatattggattgaagtttgaaattgcatttgaagtccgtcccagaagcagttccggaaccctggtccacggccacagtgtcaatggggagtacctaaatgttcacatgaaaaatggacaggtcatagtgaaagtcaataatggcatcagagatttttccacctcagtaacacccaagcagagtctctgtgatggcagatggcacagaattacagttattagagattctaatgtggttcagttggatgtggactctgaagtgaaccatgtggttggacccctgaatccaaaaccaattgatcacagggagcctgtgtttgttggaggtgttccagaatctctactgacaccacgcttggcccccagcaaacccttcacaggctgcatacgccactttgtgattgatggacacccagtgagcttcagtaaagcagccctggtcagcggcgccgtaagcatcaactcctgtccagcagcctgacatgacagagcacagctgcccaaatacaaagttctttagagcactgaaagaaacacaaagccagccaggaggaacagtaactcttccttcgggtggaagctttcatcgagttgaacaggacttaaacgaatcatcagggaccggatatttcttatttctcatttggattcttaaccttgaatccaaagtgtctgcaatggacaacaattgaaggagaggcaaacttacttgtattgagagcacacgcaattcctactggtgaaattactgtttctgtttctaataaaatagaagggattccaaataaaaaaaaaaaaaaaaaaaaaaaaa

SEQ ID NO: 4 - protein, 1816 aa, Homo sapien
  X in position 1112 = R/P
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNHVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVGSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSSGXVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 5 - protein, 296 aa, Homo sapien
  X in position 195 =R/P
SSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSSGXVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTET

SEQ ID NO: 6 - protein, 296 aa, Homo sapien; identical to SEQ ID NO: 5
  X in position 195 = R/P

SEQ ID NO: 7 - DNA, 22 bp, Artificial Sequence
  Description of Artificial Sequence PRIMER
ccaagcccgtcggatcctggcc

SEQ ID NO: 8 - DNA, 24 bp, Homo sapien
  Description of Artificial Sequence PRIMER
caatttactcgagcagacagaaac

SEQ ID NO: 9 - DNA, 3971 bp, Homo sapien
gctttcaggcgatctggagaaagaacggcagaacacacagcaaggaaaggtcctttctggggatcaccccattggctgaagatgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcccagaatgatgtgaaccctgtctctctgcagctggacctggacaggagattccagcttcaagaagtcatgatggagttccaggggcccatgcccgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggcccctccaccgctgtgcaggtccacgatgtctgtgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtgccaggggctccctgtgacccagtgaccgggcagtgtgtgtgcaaggagcatgtgcagggagagcgctgtgacctatgcaagccgggcttcactggactcacctacgccaacccgcagggctgccaccgctgtgactgcaacatcctggggtcccggagggacatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactcccctcagcccacagtgcaaccagttcacagggcagtgccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgcaatcgctacccggtgtgcgtggcctgccacccttgcttccagacctatgatgcggacctccgggagcaggccctgcgctttggtagactccgcaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggctggagatgtcttcgttgcctgacctgacacccaccttcaacaagctctgtggcaactccaggcagatggcttgcaccccaatatcatgccctggtgagctatgtccccaagacaatggcacagcctgtggctcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaagatgtggttgggaacctgcggcaggggacagtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcggcggacctgacaggactggagaagcgtgtggagcagatccgtgaccacatcaatgggcgcgtgctctactatgccacctgcaagtgatgctacagcttccagcccgttgccccactcatctgccgcctttgcttttggttgggggcagattgggttggaatgctttccatctccaggagactttcatgcagcctaaagtacagcctggaccacccctggtgtgtagctagtaagattaccctgagctgcagctgagcctgagccaatgggacagttacacttgacagacaaagatggtggagattggcatgccattgaaactaagagctctcaagtcaaggaagctgggctgggcagtatcccccgcctttagttctccactggggaggaatcctggaccaagcacaaaaacttaacaaaagtgatgtaaaaatgaaaagccaaataaaaatctttgg

SEQ ID NO: 10 - protein, 1172 aa, Homo sapien
MRPFFLLCFALPGLLHAQQACSRGACYPPVGDLLVGRTRFLRASSTCGLTKPETYCTQYGEWQMKCCKCDSRQPHNYYSHRVENVASSSGPMRWWQSQNDVNPVSLQLDLDRRFQLQEVMMEFQGPMPAGMLIERSSDFGKTWRVYQYLAADCTSTFPRVRQGRPQSWQDVRCQSLPQRPNARLNGGKVQLNLMDLVSGIPATQSQKIQEVGEITNLRVNFTRLAPVPQRGYHPPSAYYAVSQLRLQGSCFCHGHADRCAPKPGASAGPSTAVQVHDVCVCQHNTAGPNCERCAPFYNNRPWRPAEGQDAHECQRCDCNGHSETCHFDPAVFAASQGAYGGVCDNCRDHTEGKNCERCQLHYFRNRRPGASIQETCISCECDPDGAVPGAPCDPVTGQCVCKEHVQGERCDLCKPGFTGLTYANPQGCHRCDCNILGSRRDMPCDEESGRCLCLPNVVGPKCDQCAPYHWKLASGQGCEPCACDPHNSPQPTVQPVHRAVPCREGFGGLMCSAAAIRQCPDRTYGDVATGCRACDCDFRGTEGPGCDKASGRCLCRPGLTGPRCDQCQRGYCNRYPVCVACHPCFQTYDADLREQALRFGRLRNATASLWSGPGLEDRGLASRILDAKSKIEQIRAVLSSPAVTEQEVAQVASAILSLRRTLQGLQLDLPLEEETLSLPRDLESLDRSFNGLLTMYQRKREQFEKISSADPSGAFRMLSTAYEQSAQAAQQVSDSSRLLDQLRDSRREAERLVRQAGGGGGTGSPKLVALRLEMSSLPDLTPTFNKLCGNSRQMACTPISCPGELCPQDNGTACGSRCRGVLPRAGGAFLMAGQVAEQLRGFNAQLQRTRQMIRAAEESASQIQSSAQRLETQVSASRSQMEEDVRRTRLLIQQVRDFLTDPDTDAATIQEVSEAVLALWLPTDSATVLQKMNEIQAIAARLPNVDLVLSQTKQDIARARRLQAEAEEARSRAHAVEGQVEDVVGNLRQGTVALQEAQDTMQGTSRSLRLIQDRVAEVQQVLRPAEKLVTSMTKQLGDFWTRMEELRHQARQQGAEAVQAQQLAEGASEQALSAQEGFERIKQKYAELKDRLGQSSMLGEQGARIQSVKTEAEELFGETMEMMDRMKDMELELLRGSQAIMLRSADLTGLEKRVEQIRDHINGRVLYYATCK

SEQ ID NO: 11 - DNA, 7923 bp, Homo sapien
gcgcactcgggcacgcgctcggaagtcgggggtcggcgcggagtgcaggctgctcccggggtaggtgagggaagcgcggaggcggggcgcgggggcagtggtcggcgagcagcgcggtcctcgctaggggcgcccacccgtcagtctctccggcgcgagccgccgccaccgcccgcgccggagtcaggcccctgggcccccaggctcaagcagcgaagcggcctccgggggacgccgctaggcgagaggaacgcgccggtgcccttgccttcgccgtgacccagcgtgcgggcggcgggatgagagggagccatcgggccgcgccggccctgcggccccgggggcggctctggcccgtgctggccgtgctggcggcggccgccgcggcgggctgtgcccaggcagccatggacgagtgcacggacgagggcgggcggccgcagcgctgcatgcccgagttcgtcaacgccgctttcaacgtgactgtggtggccaccaacacgtgtgggactccgcccgaggaatactgtgtgcagaccggggtgaccggggtcaccaagtcctgtcacctgtgcgacgccgggcagccccacctgcagcacggggcagccttcctgaccgactacaacaaccaggccgacaccacctggtggcaaagccagaccatgctggccggggtgcagtaccccagctccatcaacctcacgctgcacctgggaaaagcttttgacatcacctatgtgcgtctcaagttccacaccagccgcccggagagctttgccatttacaagcgcacacgggaagacgggccctggattccttaccagtactacagtggttcctgcgagaacacctactccaaggcaaaccgcggcttcatcaggacaggaggggacgagcagcaggccttgtgtactgatgaattcagtgacatttctcccctcactgggggcaacgtggccttttctaccctggaaggaaggcccagcgcctataactttgacaatagccctgtgctgcaggaatgggtaactgccactgacatcagagtaactcttaatcgcctgaacacttttggagatgaagtgtttaacgatcccaaagttctcaagtcctattattatgccatctctgattttgctgtaggtggcagatgtaaatgtaatggacacgcaagcgagtgtatgaagaacgaatttgataagctggtgtgtaattgcaaacataacacatatggagtagactgtgaaaagtgtcttcctttcttcaatgaccggccgtggaggagggcaactgcggaaagtgccagtgaatgcctgccctgtgattgcaatggtcgatcccaggaatgctacttcgaccctgaactctatcgttccactggccatgggggccactgtaccaactgccaggataacacagatggcgcccactgtgagaggtgccgagagaacttcttccgccttggcaacaatgaagcctgctcttcatgccactgtagtcctgtgggctctctaagcacacagtgtgatagttacggcagatgcagctgtaagccaggagtgatgggggacaaatgtgaccgttgccagcctggattccattctctcactgaagcaggatgcaggccatgctcttgtgatccctctggcagcatagatgaatgtaatgttgaaacaggaagatgtgtttgcaaagacaatgtcgaaggcttcaattgtgaaagatgcaaacctggattttttaatctggaatcatctaatcctcggggttgcacaccctgcttctgctttgggcattcttctgtctgtacaaacgctgttggctacagtgtttattctatctcctctacctttcagattgatgaggatgggtggcgtgcggaacagagagatggctctgaagcatctctcgagtggtcctctgagaggcaagatatcgccgtgatctcagacagctactttcctcggtacttcattgctcctgcaaagttcttgggcaagcaggtgttgagttatggtcagaacctctccttctcctttcgagtggacaggcgagatactcgcctctctgccgaagaccttgtgcttgagggagctggcttaagagtatctgtacccttgatcgctcagggcaattcctatccaagtgagaccactgtgaagtatgtcttcaggctccatgaagcaacagattacccttggaggcctgctcttaccccttttgaatttcagaagctcctaaacaacttgacctctatcaagatacgtgggacatacagtgagagaagtgctggatatttggatgatgtcaccctggcaagtgctcgtcctgggcctggagtccctgcaacttgggtggagtcctgcacctgtcctgtgggatatggagggcagttttgtgagatgtgcctctcaggttacagaagagaaactcctaatcttggaccatacagtccatgtgtgctttgcgcctgcaatggacacagcgagacctgtgatcctgagacaggtgtttgtaactgcagagacaatacggctggcccgcactgtgagaagtgcagtgatgggtactatggagattcaactgcaggcacctcctccgattgccaaccctgtccgtgtcctggaggttcaagttgtgctgttgttcccaagacaaaggaggtggtgtgcaccaactgtcctactggcaccactggtaagagatgtgagctctgtgatgatggctactttggagaccccctgggtagaaacggccctgtgagactttgccgcctgtgccagtgcagtgacaacatcgatcccaacgcagttggaaattgcaatcgcttgacgggagaatgcctgaagtgcatctataacactgctggcttctattgtgaccggtgcaaagacggattttttggaaatcccctggctcccaatccagcagacaaatgcaaagcctgcaattgcaatccgtatgggaccatgaagcagcagagcagctgtaaccccgtgacggggcagtgtgaatgtttgcctcacgtgactggccaggactgtggtgcttgtgaccctggattctacaatctgcagagtgggcaaggctgtgagaggtgtgactgccatgccttgggctccaccaatgggcagtgtgacatccgcaccggccagtgtgagtgccagcccggcatcactggtcagcactgtgagcgctgtgaggtcaaccactttgggtttggacctgaaggctgcaaaccctgtgactgtcatcctgagggatctctttcacttcagtgcaaagatgatggtcgctgtgaatgcagagaaggctttgtgggaaatcgctgtgaccagtgtgaagaaaactatttctacaatcggtcttggcctggctgccaggaatgtccagcttgttaccggctggtaaaggataaggttgctgatcatagagtgaagctccaggaattagagagtctcatagcaaaccttggaactggggatgagatggtgacagatcaagccttcgaggatagactaaaggaagcagagagggaagttatggacctccttcgtgaggcccaggatgtcaaagatgttgaccagaatttgatggatcgcctacagagagtgaataacactctgtccagccaaattagccgtttacagaatatccggaataccattgaagagactggaaacttggctgaacaagcgcgtgcccatgtagagaacacagagcggttgattgaaatcgcatccagagaacttgagaaagcaaaagtcgctgctgccaatgtgtcagtcactcagccagaatctacaggggacccaaacaacatgactcttttggcagaagaggctcgaaagcttgctgaacgtcataaacaggaagctgatgacattgttcgagtggcaaagacagccaatgatacgtcaactgaggcatacaacctgcttctgaggacactggcaggagaaaatcaaacagcatttgagattgaagagcttaataggaagtatgaacaagcgaagaacatctcacaggatctggaaaaacaagctgcccgagtacatgaggaggccaaaagggccggtgacaaagctgtggagatctatgccagcgtggctcagctgagccctttggactctgagacactggagaatgaagcaaataacataaagatggaagctgagaatctggaacaactgattgaccagaaattaaaagattatgaggacctcagagaagatatgagagggaaggaacttgaagtcaagaaccttctggagaaaggcaagactgaacagcagaccgcagaccaactcctagcccgagctgatgctgccaaggccctcgctgaagaagctgcaaagaagggacgggataccttacaagaagctaatgacattctcaacaacctgaaagattttgataggcgcgtgaacgataacaagacggccgcagaggaggcactaaggaagattcctgccatcaaccagaccatcactgaagccaatgaaaagaccagagaagcccagcaggccctgggcagtgctgcggcggatgccacagaggccaagaacaaggcccatgaggcggagaggatcgcaagcgctgtccaaaagaatgccaccagcaccaaggcagaagctgaaagaacttttgcagaagttacagatctggataatgaggtgaacaatatgttgaagcaactgcaggaagcagaaaaagagctaaagagaaaacaagatgacgctgaccaggacatgatgatggcagggatggcttcacaggctgctcaagaagccgagatcaatgccagaaaagccaaaaactctgttactagcctcctcagcattattaatgacctcttggagcagctggggcagctggatacagtggacctgaataagctaaacgagattgaaggcaccctaaacaaagccaaagatgaaatgaaggtcagcgatcttgataggaaagtgtctgacctggagaatgaagccaagaagcaggaggctgccatcatggactataaccgagatatcgaggagatcatgaaggacattcgcaatctggaggacatcaggaagaccttaccatctggctgcttcaacaccccgtccattgaaaagccctagtgtctttagggctggaaggcagcatccctctgacaggggggcagttgtgaggccacagagtgccttgacacaaagattacatttttcagacccccactcctctgctgctgtccatcactgtccttttgaaccaggaaaagtcacagagtttaaagagaagcaaattaaacatcctgaatcgggaacaaagggttttatctaataaagtgtctcttccatcacgttgctaccttacccacacttccctctgatttgcgtgaggacgtggcatcctacttacgtacgtggcataacacatcgtgtgagcccatgtatgctggggtagagcaagtagccctcccctgtctcatcgatccagcagaacctcctcagtctcagtactcttgtttctataaggaaaagttttgctactaacagtagcattgtgatggccagtatatccagtccatggataaagaaaatgcatctgcatctcctgcccctcttccttctaagcaaaaggaaataaacatcctgtgccaaaggtattggtcatttagaatgtcggtagccatccatcagtgcttttagctattatgagtgtaggacactgagccatccgtgggtcaggatgcaattatttataaaagtccccaggtgaacatggctgaagatttttctagtatattaataattgactaggaagatgaactttttttcagatctttgggcagctgataatttaaatctggatgggcagcttgcactcaccaatagaccaaaagacatcttttgatattcttataaatggaacttacacagaagaaatagggatatgataaccactaaagttttgttttcaaaatcaaactaattcttacagcttttttattagttagtcttggaactagtgttaagtatctggcagagaacagttaatccctaaggtcttgacaaaacagaagaaaaacaagcctcctcgtcctagtcttttctagcaaagggataaaacttagatggcagcttgtactgtcagaatcccgtgtatccatttgttcttctgttggagagatgagacatttgacccttagctccagttttcttctgatgtttccatcttccagaatccctcaaaaaacattgtttgccaaatcctggtggcaaatacttgcactcagtatttcacacagctgccaacgctatcgagttcctgcactttgtgatttaaatccactctaaaccttccctctaagtgtagagggaagacccttacgtggagtttcctagtgggcttctcaacttttgatcctcagctctgtggttttaagaccacagtgtgacagttccctgccacacacccccttcctcctaccaacccacctttgagattcatatatagcctttaacactatgcaactttgtactttgcgtagcaggggctggggtggggggaaagaaacctattatcatggacacactggtgctattaattatttcaaatttatatttttgtgtgaatgttttgtgttttgtttatccatgctatagaacaaggaatttatgtagatatacttagtcctatttctagaatgacactctgttcactttgctcaatttttcctcttcactggcacaagtatctgaatacctccttccctcccttctagagttctttggattgtactccaaagaattgtgccttgtgtttgcagcatctccattctctaaattaatataattgctttcctccacacccagccacgtaaagaggtaacttgggtcctcttccattgcagtcctgatgatcctaacctgcagcacggtggttttacaatgttccagagcaggaacgccaggttgacaagctatggtaggattaggaaagtttgctgaagaggatctttgacgccacagtgggactagccaggaatgagggagaaatgccctttttggcaattgttggagctggataggtaagttttataagggagtacattttgactgagcacttagggcatcaggaacagtgctacttactggtgggtagactgggagaggtggtgtaacttagttcttgatgatcccacttcctgtttccatctgcttgggatataccagagtttaccacaagtgttttgacgatatactcctgagctttcactctgctggcttctcccaggcctcttctactatggcaggagatgtggtgtgctgttgcaaagttttcacgtcatcgtttcctggctagttcatttcattaagtggctacatcctaacatatgcattggtcaaggttgcagcaagaggactgaagattgactgccaagctagtttgggtgaagttcactccagcaagtctcaggccacaatggggtggtttggtttggtttccttttaactttctttttgttatttgcttttctcctccacctgtgtggtatattttttaagcagaattttattttttaaaataaaaggttctttacaagatgataccttaattacactcccgcaacacagccattattttattgtctagctccagttatctgtattttatgtaatgtaattgacaggatggctgctgcagaatgctggttgacacagggattattatactgctatttttccctgaattcttttccttggaattccaactgtggaccttttatatgtgccttcactttagctgtttgccttactctacagccttgctctccggggtggttaataaaatgcaacacttggcatttttatgttataagaaaaacagtattttatttataataaaatctgaatattttgtaacccttta

SEQ ID NO: 12 - protein, 1609 aa, Homo sapien
MRGSHRAAPALRPRGRLWPVLAVLAAAAAAGCAQAAMDECTDEGGRPQRCMPEFVNAAFNVTVVATNTCGTPPEEYCVQTGVTGVTKSCHLCDAGQPHLQHGAAFLTDYNNQADTTWWQSQTMLAGVQYPSSINLTLHLGKAFDITYVRLKFHTSRPESFAIYKRTREDGPWIPYQYYSGSCENTYSKANRGFIRTGGDEQQALCTDEFSDISPLTGGNVAFSTLEGRPSAYNFDNSPVLQEWVTATDIRVTLNRLNTFGDEVFNDPKVLKSYYYAISDFAVGGRCKCNGHASECMKNEFDKLVCNCKHNTYGVDCEKCLPFFNDRPWRRATAESASECLPCDCNGRSQECYFDPELYRSTGHGGHCTNCQDNTDGAHCERCRENFFRLGNNEACSSCHCSPVGSLSTQCDSYGRCSCKPGVMGDKCDRCQPGFHSLTEAGCRPCSCDPSGSIDECNVETGRCVCKDNVEGFNCERCKPGFFNLESSNPRGCTPCFCFGHSSVCTNAVGYSVYSISSTFQIDEDGWRAEQRDGSEASLEWSSERQDIAVISDSYFPRYFIAPAKFLGKQVLSYGQNLSFSFRVDRRDTRLSAEDLVLEGAGLRVSVPLIAQGNSYPSETTVKYVFRLHEATDYPWRPALTPFEFQKLLNNLTSIKIRGTYSERSAGYLDDVTLASARPGPGVPATWVESCTCPVGYGGQFCEMCLSGYRRETPNLGPYSPCVLCACNGHSETCDPETGVCNCRDNTAGPHCEKCSDGYYGDSTAGTSSDCQPCPCPGGSSCAVVPKTKEVVCTNCPTGTTGKRCELCDDGYFGDPLGRNGPVRLCRLCQCSDNIDPNAVGNCNRLTGECLKCIYNTAGFYCDRCKDGFFGNPLAPNPADKCKACNCNPYGTMKQQSSCNPVTGQCECLPHVTGQDCGACDPGFYNLQSGQGCERCDCHALGSTNGQCDIRTGQCECQPGITGQHCERCEVNHFGFGPEGCKPCDCHPEGSLSLQCKDDGRCECREGFVGNRCDQCEENYFYNRSWPGCQECPACYRLVKDKVADHRVKLQELESLIANLGTGDEMVTDQAFEDRLKEAEREVMDLLREAQDVKDVDQNLMDRLQRVNNTLSSQISRLQNIRNTIEETGNLAEQARAHVENTERLIEIASRELEKAKVAAANVSVTQPESTGDPNNMTLLAEEARKLAERHKQEADDIVRVAKTANDTSTEAYNLLLRTLAGENQTAFEIEELNRKYEQAKNISQDLEKQAARVHEEAKRAGDKAVEIYASVAQLSPLDSETLENEANNIKMEAENLEQLIDQKLKDYEDLREDMRGKELEVKNLLEKGKTEQQTADQLLARADAAKALAEEAAKKGRDTLQEANDILNNLKDFDRRVNDNKTAAEEALRKIPAINQTITEANEKTREAQQALGSAAADATEAKNKAHEAERIASAVQKNATSTKAEAERTFAEVTDLDNEVNNMLKQLQEAEKELKRKQDDADQDMMMAGMASQAAQEAEINARKAKNSVTSLLSIINDLLEQLGQLDTVDLNKLNEIEGTLNKAKDEMKVSDLDRKVSDLENEAKKQEAAIMDYNRDIEEIMKDIRNLEDIRKTLPSGCFNTPSIEKP

SEQ ID NO: 13 - protein, 289 aa, Homo sapien
  X in position 195 = R/P
SWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSSGXVHLEDTLKKAQINDAKYHEISIIYHNDKKMILVVDRRHVKSMDNEKMKIPFTDIYIGGAPPEILQSRALRAHLPLDINFRGCMKGFQFQKKDFNL